(19) United States
(12) Patent Application Publication
(10) Pub. No.: US 2006/0024745 A1
(43) Pub. Date: Feb. 2, 2006

(54) Variants of factor XllA

(76) Inventor: David John Pritchard, Scotland (GB)

Correspondence Address: Leon R Yankwich;Yankwich & Associates, 201 Broadway, Cambridge, MA, 02139, US

(21) Appl. No.: 10/539,720

(22) PCT Filed: Dec. 22, 2003
(86) PCT No.: PCT/GB03/05612

§ 371 Date: Jul. 18, 2005

(30) Foreign Application Priority Data
Dec. 20, 2002 (GB) 0229837.0
Dec. 20, 2002 (GB) 0229835.4
Dec. 20, 2002 (GB) 0229832.1
Dec. 20, 2002 (GB) 0229828.9

(51) Int. Cl. G01N 33/53

(52) U.S. Cl. 435/7.1

(57) ABSTRACT

Factor XIIa (activated Factor XII) exists in a variety of forms in the blood. Measurement of different forms provides information relevant for diagnosing, monitoring, or predicting the susceptibility to, progress of, or outcome of a disease or disorder, or of treatment of the disease or disorder in a subject having or suspected of having the disease or disorder.

The present invention relates to Factor XIIa, a component of the “contact activation system”.

### BACKGROUND OF THE INVENTION

Factor XIIa is an inactive zymogen present in normal blood. It is readily converted, in vitro, in the presence of kallikrein, high molecular weight kininogen and a negatively charged surface into a form, Factor XIIa, that is enzymatically active. In vitro, two forms of XIIa have previously been reported. The 80 Kd form of the serine proteinase, often called Factor αXIIa, has a 52 Kd heavy chain linked by a disulphide bond to a 28 Kd light chain. Proteolysis of this factor releases a peptide from the heavy chain, and results in a product, Factor βXIIa, that retains serine protease activity, but in which the 28 Kd chain of Factor αXIIa is disulphide-linked to a small peptide fragment derived from the former 52-Kd heavy chain. In many cases the small peptide fragment has a molecular weight of about 1000 d, but fragments of different size have been observed in vitro.

WO90/08835 discloses an immunoassay for Factor XIIa. WO 90/08835 also discloses monoclonal antibodies 2/215 and 201/9, which bind to Factor XIIa, and methods for their production. Monoclonal antibody (mAb) 2/215 is produced by hybridoma 2/215, deposited at the European Collection of Animal Cell Cultures, Divisional of Biologics, PHLS Centre for Applied Microbiology and Research, Porton Down, Salisbury SP4 0JG, England (known as ECACC) on 16 Jan. 1990 under the deposit number 90011606, and hybridoma 201/9, producing monoclonal antibody 201/9, was deposited at ECACC on 18 Jan. 1990 under deposit number 90011893.

Factor XIIa has long been known to be involved in the contact system of blood coagulation in vivo. More recent work indicates that Factor XIIa is also involved in other systems, including fibrinolysis, kininogensis, and also complement activation and angiogenesis. Many clinical and experimental data are accumulating to suggest that the contact system extends beyond haemocoagulation and that it has a role in maintaining vascular wholeness and blood pressure, that it influences various functions of endothelial cells and that it is involved in control of fibrinolysis and in maintaining the constitutive anticoagulant character of the intravascular space. Further clinical and experimental studies indicate that the contact system is involved in acute and chronic inflammation, shock of different aetiologies, diabetes, allergy, thrombo-haemorrhagic disorders including disseminated intravascular blood coagulation, and oncological diseases. Such conditions, include sepsis, spontaneous abortion and thromboembolism. In addition, Factor XIIa may be involved in tissue defence and repair. Yarovaya et al. (Yarovaya, G. A., Blokhina, T. B. & Neshkova, E. A. Contact system. New concepts on activation mechanisms and bioregulatory functions. Biochemistry (Mosc). 2002 January; 67(1):13-24) is a recent review of the contact system and new concepts on activation mechanisms and bioregulatory functions.

### SUMMARY OF THE INVENTION

The present invention provides a method for detecting or determining one or more forms of Factor XIIa in a sample, which comprises carrying out a procedure that is capable of detecting or determining the form or forms of Factor XIIa under investigation in preference to other forms of Factor XIIa.

In one embodiment, a method of the invention comprises detecting or determining the form or forms of Factor XIIa under investigation by means of an assay that enables determination of the form or forms of Factor XIIa under investigation in preference to other forms of Factor XIIa,

In another embodiment, a method of the invention comprises separating the form or forms of Factor XIIa under investigation from other forms of Factor XIIa and detecting or determining the separated form or forms of Factor XIIa.

The detection or determination of the separated form or forms of Factor XIIa may be by means of an assay that enables determination of the form or forms of Factor XIIa under investigation in preference to other forms of Factor XIIa.

In a further embodiment, a method of the invention comprises contacting the sample with a labeled antibody that is capable of binding to the form or forms of Factor XIIa under investigation and that is optionally also capable of binding to other forms of Factor XIIa, separating the form or forms of Factor XIIa under investigation from other form, and detecting or determining the form or forms of Factor XIIa under investigation.

The present invention also provides a method for diagnosing, monitoring, or predicting the susceptibility to, progress of, or outcome of a disease or disorder, or of treatment of the disease or disorder in a subject having or suspected of having the disease or disorder, which comprises detecting or determining one or more forms of Factor XIIa in preference to other forms of Factor XIIa in a sample obtained from the subject, and comparing the results obtained for the subject with the results obtained using the same assay for samples obtained from at least any one or more of the following:
(i) subjects having the disease or disorder;
(ii) subjects having the disease or disorder, which subjects were monitored in relation to the progress and/or outcome of the disease or disorder;
(iii) subjects having the disease or disorder and the treatment;
(iv) subjects having the disease or disorder and the treatment, which subjects were monitored in relation to the treatment in relation to the progress and/or outcome of the disease or disorder;
(v) subjects who do not have the disease or disorder;
(vi) the same subject before the onset of the disease or disorder or before the start of the treatment of the disease or disorder; and
(vii) the same subject at an earlier or later stage of the disease or disorder or the treatment of the disease or disorder or before the onset of the disease or disorder.

The present invention further provides a method comprising carrying out a series of assays for Factor XIIa on samples obtained from subjects having a disease or disorder or treatment for a disease or disorder, and selecting an assay that provides information on Factor XIIa levels that is relevant to the disease or disorder or the treatment.

The present invention also provides a method for providing an assay for Factor XIIa suitable for providing information relevant for diagnosing, monitoring, or predicting the susceptibility to, progress of, or outcome of a disease or disorder, or of treatment of the disease or disorder in a subject having or suspected of having the disease or disorder, which comprises carrying out a series of assays for Factor XIIa on samples obtained from subjects having the disease or disorder or the treatment, and determining which assay(s) provide information on Factor XIIa levels that is relevant to diagnosing, monitoring, or predicting the susceptibility to, progress of, or outcome of the disease or disorder, or of treatment of the disease or disorder.

The method preferably comprises comprising comparing the results obtained for Factor XIIa in the samples obtained from subjects having the disease or disorder or the treatment with the results obtained using the same assay for samples obtained from at least any one or more of the following:
(i) subjects having the disease or disorder;
(ii) subjects having the disease or disorder, which subjects were monitored in relation to the progress and/or outcome of the disease or disorder;
(iii) subjects having the disease or disorder and the treatment;
(iv) subjects having the disease or disorder and the treatment, which subjects were monitored in relation to the treatment in relation to the progress and/or outcome of the disease or disorder;
(v) subjects who do not have the disease or disorder;
(vi) the same subject before the onset of the disease or disorder or before the start of the treatment of the disease or disorder; and
(vii) the same subject at an earlier or later stage of the disease or disorder or the treatment of the disease or disorder or before the onset of the disease or disorder.

### BRIEF DESCRIPTION OF THE FIGURES

FIG. 1 shows a diagrammatic representation of the hypothesis pertaining to different forms of Factor XIIa that exist in vivo.

FIGS. 2a to 2d show HPLC traces using fluoresecence detection: FIG. 2a, plasma sample only; FIG. 2b, FITC labelled 2/215 antibody; FIG. 2c, plasma incubated with FITC labelled 2/215 antibody; FIG. 2d, trace shown in FIG. 2c after subtraction of traces shown in Figures FIGS. 2a and 2b.

FIG. 3 shows radioactivity in plasma incubated with radiolabelled 2/215 Fab fragment, after separation of components using HPLC. Peaks 1 to 5 are the result of mAb 2/215 Fab binding to plasma components, peak 6 is the remaining unbound mAb 2/215 Fab.

FIGS. 4a and 4b show the normalised response for three different sample types, namely cell rich plasma, cell poor plasma and washed cells, on a microtitre plate Factor XIIa immunoassay, using mAb 2/215 as capture antibody and labeled polyclonal antibody (polyclonal conjugate) (diamonds in FIG. 4a, spotted bars in FIG. 4b), and labeled mAb 2/215 (2/215 conjugate) (squares in FIG. 4a, black bars in FIG. 4b) for detection of cellular Factor XIIa.

FIG. 5 shows the protocol used to carry out an immunoassay for cellular factor XIIa using the IMx system of Abbott Laboratories

FIGS. 6a and 6b show the response for three different sample types, namely cell rich plasma, cell poor plasma and cell suspension, on a microtitre plate Factor XIIa immunoassay, using mAb 2/215 as capture antibody and labeled polyclonal antibody (polyclonal conjugate) (diamonds in FIG. 6a, spotted bars in FIG. 6b) and labeled mAb 2/215 (2/215 conjugate) (squares in FIG. 6a, black bars in FIG. 6b) for detection of cellular Factor XIIa.

FIGS. 7a and 7b show the response for three different sample types, cell rich plasma, cell poor plasma and cell suspension, in an IMx Factor XIIa immunoassay, using mAb 2/215 as capture antibody and labeled mAb 201/9 (201/9 conjugate) (diamonds in FIG. 7a, spotted bars in FIG. 7b) and labeled mAb 2/215 (2/215 conjugate) (squares in FIG. 7a, black bars in FIG. 7b) for detection of cellular Factor XIIa.

FIGS. 8a and 8b show flow cytometry data obtained for FITC labeled mAb 2/215 incubated with plasma. FIG. 8a shows data obtained for the plasma in the absence of labeled antibody, FIG. 8b shows data obtained when plasma was incubated with the labeled antibody. The shift in the distribution indicates that the labeled 2/215 antibody binds to a cellular component of plasma.

FIG. 9 shows the cellular Factor XIIa content of plasma for eight individuals as determined by the addition of radiolabelled mAb 2/215.

FIGS. 10a and 10b show the response for three different sample types, namely cell rich plasma, cell poor plasma and cell suspension, from an individual “totally deficient” in Factor XII. A Factor XIIa immunoassay was performed on an IMx analyser, using mAb 2/215 as capture antibody and labelled mAbs 201/9 (diamonds in FIG. 10a, spotted bars in FIG. 10b) and 2/215 (squares in FIG. 10a, black bars in FIG. 10b) as conjugates.

FIGS. 11a and 11b show the normalised response for three different sample types, namely cell rich plasma, cell poor plasma and cell suspension, from an individual “totally deficient” in Factor XII. A Factor XIIa immunoassay was performed on an IMx analyser, using mAb 2/215 as capture antibody and labeled mAbs 201/9 (diamonds in FIG. 11a, spotted bars in FIG. 11b) and 2/215 (squares in FIG. 11a, black bars in FIG. 11b) as conjugates.

FIGS. 12a and 12b show the normalised response for three different sample types, namely cell rich plasma, cell poor plasma and cell suspension, from a normal volunteer and from an individual “totally deficient” in Factor XII. Factor XIIa assays were performed on an IMx analyser, using mAb 2/215 as capture antibody and labelled mAb 2/215 as conjugate. (Diamonds in FIG. 12a and spotted bars in FIG. 12b denote samples from the normal volunteer; squares FIG. 12a and black bars in FIG. 12b denote samples from the “Factor XII deficient” individual.)

FIGS. 13a and 13b shows the normalised response for three different sample types, namely cell rich plasma, cell poor plasma and cell suspension, from an normal volunteer and from an individual totally deficient in Factor XII. A Factor XIIa immunoassay was performed on an IMx analyser, using mAb 2/215 as capture antibody and labelled mAb 201/9 as conjugate. (Diamonds in FIG. 13a and spotted bars in FIG. 13b denote samples from the normal volunteer; squares FIG. 13a and black bars in FIG. 13b denote samples from the “Factor XII deficient” individual.)

FIG. 14 shows lipid-bound Factor XIIa concentrations obtained for 12 healthy volunteers, assessed by addition of radiolabelled 2/215 antibody fragments to citrated plasma, removal of cellular material, precipitation of lipoproteins using a manganese/heparin precipitation method, and measuring radioactivity in the precipitated fraction.

FIG. 15 shows lipid-bound Factor XIIa concentrations obtained for 64 patients admitted to hospital with chest pain, assessed by addition of radiolabelled 2/215 antibody fragments to whole blood, following removal of cellular material, precipitation of lipoproteins using a phosphotungstate precipitation method, and measuring radioactivity in the precipitated fraction.

FIG. 16 shows lipid bound Factor XIIa concentrations (expressed as absorbance at 550 nm), as assessed by an ELISA method, obtained for eight volunteers.

FIGS. 17a to 17d show HPLC traces using fluorescence detection: FIG. 17a, urine sample only; FIG. 17b, FITC labelled mAb 2/215; FIG. 17c, urine incubated with FITC labeled mAb 2/215; FIG. 17d, trace shown in FIG. 17c after subtraction of traces shown in FIGS. 17a and 17b.

FIG. 18 shows radioactivity in urine incubated with radiolabelled mAb 2/215 Fab Fragment, after separation of components using HPLC. Peak 1 is the result of mAb 2/215 Fab binding to Factor XIIa in the urine, peak 2 is the remaining unbound mAb 2/215 Fab.

FIG. 19 shows a typical pattern of values obtained using two different immunoassays to determine Factor XIIa concentrations in plasma samples of patients immediately prior to, immediately after, and five days after percutaneous transluminal coronary angioplasty (PTCA). Assay 1 is an immunoassay involving a sample incubation step. The assay uses mAb 2/215 as capture antibody and labeled mAb 201/9 as conjugate and incorporates the addition of Triton in the sample incubation step. Assay 2 uses mAb 2/215 as capture antibody with anti-Factor XII polyclonal antibody as conjugate, with no Triton added during the sample incubation step. Data points represented by diamonds indicate the results obtained with Assay 1; data points represented by squares indicate results obtained with Assay 2.

FIG. 20 shows the Factor XIIa concentration in plasma samples obtained from four patients (patients S0216, S0794, S0811 and S0909) obtained by analyzing plasma samples obtained immediately prior to, immediately after, and five days after coronary angioplasty (PCTA). Factor XIIa was measured in an immunoassay involving a sample incubation step. The assay used mAb 2/215 as capture antibody and labelled anti-Factor XII polyclonal antibody as conjugate with no Triton added during the sample incubation step. The spotted bars indicate the values obtained pre-PCTA, the shaded bars indicate the values obtained post-PCTA, and the black bars indicate the values obtained 5 days post-PCTA.

FIG. 21 shows Factor XIIa concentrations of samples obtained from patients immediately prior to, immediately after and five days after thrombolytic therapy. Assay 1 is an immunoassay involving a sample incubation step. The assay uses mAb 2/215 as capture antibody and labeled mAb 201/9 as conjugate and incorporates the addition of Triton in the sample incubation step. Assay 2 uses mAb 2/215 as capture antibody with anti-Factor XII polyclonal antibody as conjugate, with no Triton added during the sample incubation step. Data points represented by diamonds indicate the results obtained with Assay 1; data points represented by squares indicate results obtained with Assay 2. The results are typical patterns of values obtained from an individual.

FIG. 22 shows Factor XIIa concentrations for three patients (S0684, S0685 and S0693), in samples taken immediately prior to, immediately after and five days after thrombolytic therapy. Factor XIIa was measured in an immunoassay involving a sample incubation step. The assay used mAb 2/215 as capture antibody and labelled anti-Factor XII polyclonal antibody as conjugate with no Triton added during the sample incubation step. The spotted bars indicate the values obtained pre-PCTA, the shaded bars indicate the values obtained post-PCTA, and the black bars indicate the values obtained 5 days post-PCTA.

FIG. 23 shows frequency of repeat troponin positive events during the hospitalization period following the initial admission of patients admitted to hospital with suspected myocardial infarction or acute coronary syndrome. The frequency of repeat troponin positive events are grouped according to the concentrations of Factor XIIa. Factor XIIa was measured in an immunoassay involving a sample incubation step. The assay used mAb 2/215 as capture antibody and the same antibody labeled with alkaline phosphatase as conjugate and incorporated the addition of Triton in the sample incubation step.

FIG. 24 shows frequency of repeat troponin positive events during the hospitalization period as in FIG. 23 using a number of different assays each preferentially measuring different forms of Factor XIIa. This demonstrates that specific forms of Factor XIIa provide clinical utility whilst others do not. The assays were immunoassays involving a sample incubation step. Forms of Factor XIIa preferentially measured in Assay a are represented by light bars incorporating dots. Assay a used mAb 2/215 (coated at 15 ug ml⁻¹ in a bicarbonate buffer) as capture antibody and the same antibody labeled with alkaline phosphatase as conjugate and incorporated the addition of Triton in the sample incubation step (as in the data shown in FIG. 23). Forms of Factor XIIa preferentially measured in Assay b are represented by dark bars. Assay b used mAb 2/215 as capture antibody (coated at 2 ug ml⁻¹ in a phosphate buffer) and mAb 201/9 labeled with alkaline phosphatase as conjugate and not incorporating the addition of Triton in the sample incubation step. Forms of Factor XIIa preferentially measured in Assay c are represented by light bars incorporating diagonal lines. Assay c used mAb 2/215 as capture antibody (coated at 2 ug ml⁻¹ in a phosphate buffer) and polyclonal antibody raised against βXIIa labeled with alkaline phosphatase as conjugate and not incorporating the addition of Triton in the sample incubation step.

FIG. 25 shows the frequency of subsequent troponin positive events within thirty days of admission date of the same patients as in FIG. 23. The frequency of repeat troponin positive events is grouped according to the concentrations of Factor XIIa. Factor XIIa was measured in an immunoassay involving a sample incubation step. The assay used mAb 2/215 as capture antibody and labeled mAb 201/9 as conjugate and incorporates the addition of Triton in the sample incubation step. The dark bars show non-fatal troponin positive events, the light bars with diagonal lines show fatal troponin positive events.

FIG. 26 FIG. 25 shows the frequency of subsequent troponin positive events within thirty days of admission date as in FIG. 25 using a number of different assays each preferentially measuring different forms of Factor XIIa. This demonstrates that specific forms of Factor XIIa provide clinical utility whilst others do not. Forms of Factor XIIa preferentially measured in Assay x are represented by light bars incorporating dots. Assay x used mAb 2/215 (coated at 2 ug ml⁻¹ in a phosphate buffer) and polyclonal antibody raised against βXIIa labeled with alkaline phosphatase as conjugate and not incorporating the addition of Triton in the sample incubation step. Forms of Factor XIIa preferentially measured in Assay y are represented by dark bars. Assay y used mAb 2/215 (coated at 2 ug ml⁻¹ in a phosphate buffer) and polyclonal antibody raised against Factor XII labeled with alkaline phosphatase as conjugate and not incorporating the addition of Triton in the sample incubation step. Forms of Factor XIIa preferentially measured in Assay Z are represented by light bars incorporating diagonal lines. Assay z used mAb 2/215 (coated at 15 ug ml⁻¹ in a bicarbonate buffer) as capture antibody and labeled mAb 201/9 as conjugate and incorporates the addition of Triton in the sample incubation step (as in the data shown in FIG. 25).

FIG. 27 shows the frequency of death as the clinical endpoint using the same sample from the patients as in FIGS. 23 and 25. Frequency of death is grouped according to concentrations of Factor XIIa. Factor XIIa was measured in an immunoassay involving a sample incubation step. The assay used mAb 2/215 as capture antibody and anti-Factor XII polyclonal antibody as conjugate with no Triton added during the sample incubation step. The dark bars indicate cardiac death with no recorded second troponin positive release. The light bars with diagonal lines indicate second troponin positive release and death.

FIG. 28 shows the frequency of death as the clinical endpoint as in FIG. 27 using a two different assays each preferentially measuring different forms of Factor XIIa. This demonstrates that specific forms of Factor XIIa provide clinical utility whilst others do not. Forms of Factor XIIa preferentially measured in Assay i are represented by light bars incorporating dots. Assay i used mAb 2/215 (coated at 2 ug ml⁻¹ in a phosphate buffer) and the same antibody labeled with alkaline phosphatase as conjugate and not incorporating the addition of Triton in the sample incubation step. Forms of Factor XIIa preferentially measured in Assay ii are represented by dark bars. Assay ii used mAb 2/215 (coated at 2 ug ml⁻¹ in a phosphate buffer) as capture antibody and anti-Factor XII polyclonal antibody as conjugate with no Triton added during the sample incubation step (as data shown in FIG. 27).

FIG. 29 shows the frequency of repeat non-fatal myocardial infarctions (troponin positive events) and cardiac deaths during the 6 month period following the initial admission of patients admitted to hospital with suspected myocardial infarction. The frequency of repeat events is grouped according to the concentrations of lipid bound Factor XIIa. The light bars indicate a non-fatal myocardial infarction, the dark bars indicate cardiac death.

FIG. 30 shows urinary Factor βXIIa concentrations for 5 healthy volunteers and 5 individuals with renal disease as ascertained by incubation with radiolabelled antibody and HPLC.

FIG. 31 shows urinary Factor βXIIa values for 5 healthy volunteers and 5 individuals with renal disease expressed as absorbance values obtained in a microtitre plate immunoassay. Factor XIIa was measured in an immunoassay involving a sample incubation step. The assay used mAb 2/215 as capture antibody and labeled mAb 201/9 as conjugate and incorporates the addition of Triton in the sample incubation step.

### DEFINITIONS

Antibody includes any antibody fragment that is capable of binding antigen, for example, Fab and F(ab′)₂ fragments, and also recombinant, chimeric and humanized antibodies.

Antibody conjugate, also detection antibody, denotes an antibody labeled with a label that is directly or indirectly analyzable.

Capture antibody denotes an antibody that is immobilized on a solid phase for use in an immunoassay.

Capture assay denotes an immunoassay in which a capture antibody immobilized on a solid phase is contacted with a sample. If the sample comprises antigen capable of binding to the immobilized antibody and if the reaction conditions are appropriate, the antigen will form an antigen-antibody complex with the immobilized antigen and hence will be “captured” on the solid phase and can subsequently be detected or determined.

Cells, unless specified otherwise, denotes intact cells, cell remnants and cellular material.

Cellular Factor XIIa and cellular Factor XII denote Factor XIIa and Factor XII, respectively, present on the surface of a cell, or bound to a cell, cell remnants or cellular material.

Detection denotes a qualitative investigation.

Detection and/or determination denotes a quantitative or semi-quantitative investigation.

Factor XIIa, also called activated Factor XII, denotes any enzymatically active form or fragment of the zymogen, Factor XII.

High affinity binding partner denotes a molecule that forms a complex with Factor XIIa, which complex cannot be disrupted by simple methods, for example, by addition of a detergent or by competition with another species.

Lipid bound Factor XIIa denotes Factor XIIa associated with lipid material, for example, in association with lipids, especially lipoproteins and remnants thereof.

Low affinity binding partner denotes a molecule that forms a complex with Factor XIIa, which complex can be readily disrupted by simple methods, for example, by addition of a detergent or by competition with another species.

Monoclonal antibody (mAb) 2/215, also called antibody 2/215, is the antibody produced by hybridoma 2/215, deposited at the European Collection of Animal Cell Cultures, Divisional of Biologics, PHLS Centre for Applied Microbiology and Research, Porton Down, Salisbury SP4 0JG, England (known as ECACC) on 16 Jan. 1990 under the deposit number 90011606.

Monoclonal antibody (mAb) 2/215 analogue denotes an antibody that has Factor XIIa binding properties that are substantially the same as those of mAb 2/215.

Monoclonal antibody (mAb) 201/9, also called antibody 201/9, is the antibody produced by hybridoma 201/9, which was deposited at ECACC on 18 Jan. 1990 under deposit number 90012512.

Monoclonal antibody (mAb) 201/9 analogue denotes an antibody that has Factor XIIa binding properties that are substantially the same as those of mAb 201/9.

Sample comprising cells denotes both samples of body fluids that comprise cells and samples of isolated cells.

Species and forms are terms that are used interchangeably in relation to Factor XIIa.

ug and ul denote micrograms and microlitres, respectively.

Urinary Factor XIIa denotes Factor XIIa present in urine.

### DETAILED DESCRIPTION OF THE INVENTION

Forms of Factor XIIa

The present invention is based on our surprising observation that Factor XIIa (activated Factor XII) exists in a variety of species or forms in the blood, and that measurement of different species or forms provides information relating to a variety of clinical conditions.

Without being bound by the following, our hypothesis pertaining to the existence of Factor XIIa in a variety of forms in vivo is as follows. Variation in forms of Factor XIIa may reflect any of the following:
(i) variations in the molecular weight and peptide chain length of Factor XIIa, such variants including Factors αXIIa, βXIIa and γXIIa;
(ii) association of two or more molecules of Factor XIIa or variants thereof according to (i), for example, in the form of a complex, in a body fluid, the Factor XIIa molecules not being bound to cellular or lipid material;
(iii) association of Factor XIIa, or a variant thereof according to (i) above, with cellular material, including cells and cell remnants or with lipids, especially lipoproteins and remnants thereof;
(iv) associated of Factor XIIa, or a variant thereof according to (i) above, with one or more other molecular species, for example, high affinity binding proteins, for example inhibitory molecules, or low affinity binding proteins.

It is also hypothesized that not all forms of Factor XIIa provide equal information pertaining to a defined medical condition and, therefore, assays that preferentially measure particular forms will provide improved clinical utility, for example, in relation to any one or more of diagnosis, prediction and monitoring of diseases and disorders and treatment thereof.

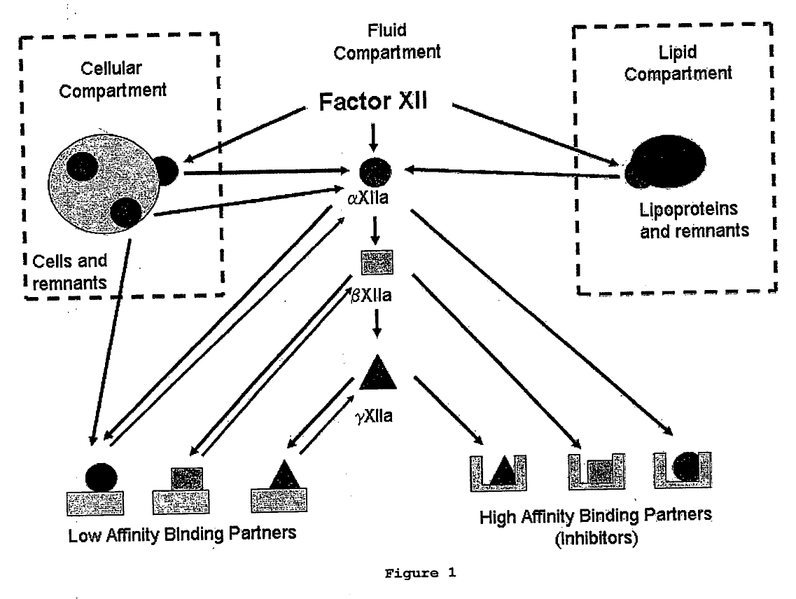

A diagrammatic representation of our hypothesis is shown in FIG. 1. Variation in forms of Factor XIIa reflecting the molecular weight and peptide chain sequence of the Factor XIIa result from progressive cleavage of the inactive zymogen Factor XII. Factor XII undergoes a cleavage resulting in an 80 Kd active serine proteinase, called Factor αXIIa and referred to as “αXIIa” in FIG. 1, that comprises a 52 Kd heavy chain linked by a disulphide bond to a 28 Kd light chain. Proteolysis of this factor releases a peptide from the heavy chain and results in a product, called Factor βXIIa and referred to as “βXIIa” in FIG. 1, that retains serine protease activity, but in which the 28 Kd chain of αXIIa is disulphide linked to a small peptide fragment derived from the former 52 Kd heavy chain. Factor βXIIa can undergo further proteolytic cleavage resulting in a fragment with a molecular weight of approximately 15 Kd, which we have called Factor γXIIa and referred to as “γXIIa” in FIG. 1.

Factor XIIa in any one of its variant forms, for example, as Factor αXIIa, βXIIa or γXIIa can associate with other molecular species, including high affinity binding partners, for example, inhibitors, for example, C1 esterase inhibitor, and other binding proteins, for example, low affinity binding partners. It is postulated that association of Factor XIIa with such other binding proteins, for example, low affinity binding partners, may be reversible and may hinder binding to inhibitory proteins and hence reduce or prevent inhibition of Factor XIIa activity.

Factor XIIa in any one of its variant forms, for example, as Factor αXIIa, βXIIa or γXIIa may associate with and dissociate from lipids, for example, lipoproteins, which may be in the form of particles and/or remnants of particles. Factor XIIa in any one of its variants forms, for example, as Factor αXIIa, βXIIa or γXIIa may associate with and dissociate from any of cells and cellular fragments. Particularly in the case of Factor XIIa associated with cells, cellular fragments, lipoproteins and lipoprotein remnants, several molecules of a form of Factor XIIa may be present on an individual particle. Furthermore, several molecules of Factor XIIa, either the same or different forms, may be present as a complex of Factor XIIa molecules. In the interests of clarity, such complexes are not shown in FIG. 1.

FIG. 1 shows the postulated interconversions between various forms of Factor XIIa. In the interests of clarity any interactions between Factor βXIIa and Factor γXIIa with cells and remnants thereof, and with lipoproteins and remnants thereof are not shown.

It is hypothesized that the system shown in FIG. 1 is a dynamic system. It is also postulated that different forms of Factor XIIa have different roles in physiology and pathology, and that preferential measurement of particular forms of Factor XIIa will result in improved clinical utility in diagnosis, prediction and monitoring of diseases and disorders and treatment thereof, compared with measuring undefined forms of Factor XIIa.

Cellular Factor XIIa

A number of authors have suggested that activation of Factor XII to Factor XIIa can occur on cell surfaces and have provided data to support that hypothesis. In particular authors have suggested that activation of Factor XII occurs on cells, notably endothelial cells, through the construction of multi-molecular assemblies that also contain High Molecular Weight Kininogen, Pre-kallikrein and Factor XI. These models indicate that, after it has been activated, Factor XIIa dissociates from the assembly and does not remain on the cell surface for a prolonged time, see for example, Yarovaya et al. (loc. cit.).

The present invention is based on our surprising observation that Factor XIIa exists in various forms, one of which is Factor XIIa present on the surface of cells circulating in the blood and on remnants thereof and on cellular material derived therefrom. This form of Factor XIIa is called “cellular Factor XIIa”. This observation is contrary to the previous findings described above that, after activation in a multi-molecular assembly on a cell surface, Factor XIIa dissociates from the assembly and does not remain bound to the cell.

A further observation is that, when Factor XIIa is cellular, not all Factor XIIa epitopes appear to be as accessible as when Factor XIIa is not cellular. For example, monoclonal antibody 2/215 is capable of binding effectively to cellular Factor XIIa and to non-cellular Factor XIIa. However, monoclonal antibody 201/9 and a sheep polyclonal antibody raised against Factor βXIIa do not appear to be able to bind as effectively to cellular Factor XIIa as to non-cellular Factor XIIa.

It appears that, in blood, Factor XIIa may be present in particular on granulocytes, especially a sub-population of granulocytes that, on flow cytometry, show a slightly higher scatter than other granulocytes, which indicates a different morphology from other sub-populations. These observations may have clinical implications, see below.

Lipid Bound Factor XIIa

Another aspect of our surprising observation that Factor XIIa exists in various forms is that some Factor XIIa is associated with lipids, for example, lipoproteins and remnants thereof in the blood, and that measurement of this lipid bound Factor XIIa provides information relating to a variety of clinical conditions.

Urinary Factor XIIa

A further aspect of our surprising observation that Factor XIIa exists in various forms is that Factor XIIa is present in urine, and that measurement of urinary Factor XIIa provides information relating to a variety of clinical conditions.

Molecular Complexes and Associations of Factor XIIa with other Molecular Species

Our observations indicate that two or more molecules of Factor XIIa may be associated with each other in the form of a complex, and also that Factor XIIa may be associated with one or more other molecular species, for example, high affinity binding proteins, for example inhibitory molecules, or low affinity binding proteins. The results obtained when carrying out immunoassays in the presence and absence of a detergent, which would be expected to disrupt molecular complexes of Factor XIIa and associations of Factor XIIa with low affinity binding partners but not associations with high affinity binding partners, also indicate the presence of molecular complexes and associations with binding partners.

Detection and/or Determination of Different Forms of Factor XIIa

The present invention provides a method for detecting or determining one or more forms of Factor XIIa in a sample, which comprises carrying out a procedure that is capable of detecting or determining the form or forms of Factor XIIa under investigation in preference to other forms of Factor XIIa.

In one embodiment, a method of the invention comprises detecting or determining the form or forms of Factor XIIa under investigation by means of an assay that enables determination of the form or forms of Factor XIIa under investigation in preference to other forms of Factor XIIa.

In another embodiment, a method of the invention comprises separating the form or forms of Factor XIIa under investigation from other forms of Factor XIIa and detecting or determining the separated form or forms of Factor XIIa.

The detection or determination of the separated form or forms of Factor XIIa may be by means of an assay that enables determination of the form or forms of Factor XIIa under investigation in preference to other forms of Factor XIIa.

In a further embodiment, a method of the invention comprises contacting the sample with a labeled antibody that is capable of binding to the form or forms of Factor XIIa under investigation and that is optionally also capable of binding to other forms of Factor XIIa, separating the form or forms of Factor XIIa under investigation from other form, and detecting or determining the form or forms of Factor XIIa under investigation.

According to the invention, therefore, the form or forms of Factor XIIa under investigation may first be separated from other forms of Factor XIIa and then the Factor XIIa may be determined. A general assay for Factor XIIa may be used i.e. an assay that is not specific for any particular form of Factor XIIa, but it may be advantageous to use an assay that enables determination of form or forms of Factor XIIa under investigation in preference to other forms of Factor XIIa. Examples of such assays are given below. Such a procedure may be used to detect or determine, for example, cellular Factor XIIa, molecular complexes and associations of Factor XIIa with other molecular species.

Alternatively, an assay that enables determination of form or forms of Factor XIIa under investigation in preference to other forms of Factor XIIa may be carried out directly on a sample without previous separation of different forms of Factor XIIa. Examples of such assays are given below. Such an assay may be carried out directly on a sample. Such a procedure may be used to detect or determine, for example, molecular complexes and associations of Factor XIIa with other molecular species.

As a further alternative, a sample comprising forms of Factor XIIa may be contacted with a labeled antibody and then separation of the form or forms of Factor XIIa under investigation may be carried out, with detection or determination of the separated forms. Such a procedure may be used to detect or determine, for example, lipid bound Factor XIIa.

Separation of Forms of Factor XIIa

Forms of Factor XIIa may be separated on the basis of their physical, chemical or immunological properties. Any such separation should generally be carried under conditions such that the form or forms of Factor XIIa under investigation are maintained unchanged, for example, the conditions should generally be such that any complexes or molecular associations are not disrupted, and that any form of Factor XIIa bound to another material, for example, to cellular or lipid material, is not released from that material. However, in some circumstances it may be desired to release Factor XIIa from an association or from material to which it is bound.

Separation on the Basis of Physical Properties

Different forms of Factor XIIa may be separated on the basis of molecular weight, for example, using chromatographic procedures, for example, High Pressure Liquid Chromatography (HPLC), flow cytometry or ultracentrigation techniques, followed by assessment of the separated material.

Assessment can be done in several ways, for example by use of an immunoassay on the separated forms, or by use of an enzymatic assay, for example using a chromogenic substrate such as S2302 (Kabi Diagnostics, Uxbridge, England). Antibodies against Factor XIIa may be used in conjunction with HPLC. For example, labeled antibodies may be reacted with the sample, and the resulting mixture may be subjected to HPLC separation. The complexes of antibody with particular forms of Factor XIIa can then be determined using a suitable detection system for the material used to label the antibody.

Separation of Molecular Complexes and of Associations of Factor XIIa with Binding Partners on the Basis of Physical Properties

Such a method may be useful, inter alia, for separating molecular complexes comprising two or more molecules of Factor XIIa from other forms of Factor XIIa, and also for separating forms of Factor XIIa associated with high affinity or low affinity binding partners.

It is generally preferable to carry out such separation under conditions such that Factor XIIa complexes are not disrupted and that Factor XIIa is not dissociated from a binding partner. For example, it is generally preferable to avoid the presence of detergents, which tend to disrupt complexes and some molecular associations. However, in some circumstances it may be desirable that disruption occurs. For example, if it is desired to release Factor XIIa from low affinity binding partners or to separate Factor XIIa associated with low affinity binding partners from Factor XIIa associated with high affinity binding partners, appropriate conditions, for example, a detergent, may be used, resulting in dissociation of Factor XIIa from low affinity binding partners but not from high affinity binding partners.

Separation of Cellular Factor XIIa and Lipid Bound Factor

XIIa on the basis of physical or chemical properties Cellular and lipid bound Factor XIIa may be separated from other forms of Factor XIIa by physical or chemical methods, or by combinations thereof. For example, cellular Factor XIIa may be separated by centrifugation or flow cytometry. Lipid bound Factor XIIa may be separated, for example, by lipoprotein precipitation agents and, generally, centrifugation, or by density layer ultracentrifugation.

It is generally preferable to carry out separation under conditions such that the Factor XIIa is not dissociated from the cellular or lipid material. For example, it is generally preferable to avoid the presence of detergents. However, in some circumstances it may be desirable that disruption occurs. If it is desired to separate Factor XIIa from the material to which it is bound, appropriate conditions may be used.

Immunological Separation

A form or forms of Factor XIIa under investigation may be separated from other forms by means of an immunological method using antibodies that show preferential binding for the form or forms of Factor XIIa under investigation. For example, immunoaffinity chromatography may be carried out, the antibody being immobilized on an appropriate solid support. Measurement of enzymic activity in either the bound or un-bound fractions may be carried out after chromatography. Preferred antibodies for such use are as described below in relation to immunoassays.

As described above in relation to separation on the basis of physical or chemical properties, separation by immunoaffinity chromatography should generally be carried out under conditions such that the form or forms of Factor XIIa is/are maintained unchanged, for example, complexes and associations are not disrupted and bound molecules are not released. However, there may be circumstances when disruption is desired. If so, appropriate conditions may be used.

Determining Suitability of Assays

Methods for detecting or determining Factor XIIa are known and include chromogenic, for example, amidolytic assays and various types of immunoassays, for example, as described in more detail below.

If the form or forms of Factor XIIa under investigation have been separated from other forms of Factor XIIa before the assay is carried out, an assay that does not discriminate between different forms of Factor XIIa may be used i.e. a “general” Factor XIIa assay. Even after a prior separation step it may, however, be advantageous to use an assay that is capable of detecting or determining the form or forms of Factor XIIa under investigation preferentially in relation to other forms.

If no separation step is carried out, the assay used must be capable of detecting or determining the form or forms of factor XIIa under investigation. An assay known to suitable for detecting or determining Factor XIIa may be tested for the ability to detect or determine the desired form or forms of Factor XIIa in a sample.

For example, using a sample known to comprise cellular Factor XIIa, the results obtained for an assay under investigation are compared with the results obtained using an assay known to be suitable for the detection of cellular Factor XIIa. Monoclonal antibody 2/215 is capable of binding effectively to cellular Factor XIIa. An immunoassay involving mAb 2/215 or an analogue thereof may be used as a comparison assay. The same considerations apply to other forms of Factor XIIa.

An alternative is to carry out the assay under investigation on a portion of a sample known to comprise the desired form of Factor XIIa, for example, cellular Factor XIIa. In that case, the sample should not contain non-cellular Factor XIIa. Another portion of the sample is treated to release the Factor XIIa from the cells, the treated cells are isolated, the assay is repeated, and the results of the two assays are compared. If result obtained for the assay on the sample that contains cellular Factor XIIa is higher than that obtained from the sample treated to remove the cellular Factor XIIa, that indicates that the assay is suitable for detecting or determining cellular Factor XIIa. The same considerations apply to other forms of Factor XIIa.

Specificity of an Assay for One or More Forms of Factor XIIa

Specificity of an assay for one or more forms of Factor XII relative to other forms may be achieved or improved by design of the assay. The parameters of the assay may be adjusted such that the forms or forms of Factor XIIa under investigation is/are detected or determined preferentially relative to other forms of Factor XIIa.

Such optimization of an assay is standard practice in the art, and suitable techniques are well known, see for example, Principles and Practice of Immunoassays, Eds. Price C P & Newman D J, Stockton Press, 1991.

In the case of an immunoassay, parameters that can be adjusted to achieve a desired specifity may include any one or more of choice of the antibody or combination of antibodies to be used; presence, absence and choice of a detergent; and conditions used for plate coating in the case of an antigen capture assay involving an antibody coated on a solid phase.

For example, in the case of microtitre plate immunoassays there are a number of parameters that may be altered to measure certain forms of Factor XIIa preferentially relative to other forms.

One example is in the selection of capture antibody, for example, mAb 2/215 may be used, or an alternative, for example, mAb 201/9 or mAb 2/15, which preferentially detect different forms of Factor XIIa.

The formulation of the solution used for coating the solid phase with capture antibody also affects the preferential measurement of different forms of Factor XIIa, for example, the concentration of antibody included in the formulation, and the pH and constituents of the buffer are important.

A further parameter that influences which forms are preferentially measured is the presence or absence of a detergent, for example, Triton, in the sample during incubation with the antibody. It is postulated that the presence of a detergent may disrupt complexes, for example, complexes of Factor XIIa molecules, and/or may release Factor XIIa previously bound to cells and/or lipids. The nature and/or amount of a detergent may also influence the assay.

An additional example of a parameter that can be manipulated to affect the preferential measurement of particular forms of Factor XIIa is the choice of antibody that is labelled to form the conjugate used for detecting antigen-antibody complexes.

It should be noted that there are complex interactions between the assay parameters, for example the effect of incorporating a detergent in an assay is dependent upon the combination of capture antibody, coating antibody concentration, coating buffer, and conjugate antibody used. The optimum conditions for detecting or determining a desired form of Factor XIIa may be determined by appropriate manipulation of the various parameters, in accordance with normal practice in the art.

Samples and Sample Preparation

Samples

Measurement of different forms of Factor XIIa may be performed on a sample of a body fluid, for example, whole blood, plasma, serum, urine, cerebrospinal fluid, saliva or tears; or a sample comprising cells isolated from a body fluid, that is to say, cells substantially free from the liquid phase in which they exist in vivo; or a sample comprising tissue or cells obtained from a tissue sample.

Sample Preparation

Samples may be obtained and prepared according to normal practice, see for example, Young, D. S. & Bermes, E. W. “Specimen collection and processing” in Tietz Textbook of Clinical Chemistry 2^{nd} Edition” Eds. Burtis, C. A. & Ashwood, E. R., Saunders (1994), also Methods in Enzymology, H. Van Vunakis and J. J. Langone (Eds), 1981, 72(B); Practice and Theory of Enzyme Immunoassays, P Tijssen, Laboratory Techniques in Biochemistry and MolecuIar-Biology, R. J. Burden and P. H. Van Knippenberg (Eds), Elsevier, 1985; Introduction to Radioimmunoassay and Related Techniques, T. Chard, ibid, 3rd Edition, 1987; and Methods in Enzymology, H. Van Vunakis and J. J. Langone (Eds) 1981, 74(C).

Body Fluids

According to the present invention, one or more forms of Factor XIIa may be detected or determined in a sample of a body fluid. Examples of body fluids are whole blood, plasma, serum, urine, cerebrospinal fluid, saliva and tears. Samples of body fluid may be obtained and prepared in a conventional manner, for example, as described in the references above.

The selective measurement of particular forms of Factor XIIa in preference to other forms may be achieved as in the section on assays below.

Cellular Factor XIIa

In one embodiment, the present invention provides a method which comprises detecting or determining Factor XIIa in a sample comprising cells obtained from a mammalian subject, generally a human, particularly cells circulating in blood or another body fluid.

Measurement of cellular Factor XIIa may be performed on a sample of a body fluid, or cells may be isolated, that is to say, made substantially free from the liquid phase in which they exist in vivo, from a sample of a body fluid, for example, whole blood or plasma, prior to analysis to determine the cellular Factor XIIa. Alternatively, cells may be obtained from a tissue sample.

If the assay used is capable of detecting or determining both cellular and non-cellular Factor XIIa, carrying out the assay on a sample comprising cells will detect or determine both the cell-bond and the non-cellular analyte. However, if the assay is carried out on a sample of isolated cells, the result will be for cellular analyte only. The term “a sample comprising cells” is used herein to denote both samples of body fluids that comprise cells and samples of isolated cells.

Cells, including cell remnants and cellular material, may be isolated, for example, as described above “Separation of forms of Factor XIIa”. For example, cells may be isolated by centrifugation and washing. Preferably the cells are centrifuged and washed at least one, preferably two or more times. Centrifugation should generally be carried out under sufficiently high g forces that the cells form a discrete pellet that can be separated from the supernatant. The pellet may be washed in a suitable medium that does not affect the cellular Factor XIIa, for example, that does not cause cellular Factor XIIa to become dissociated from cells. For example, phosphate buffered saline pH7.4 may be used for washing and for suspension of cells for the detection or detection and/or determination of cellular Factor XIIa. Flow cytometry may be used to isolate cells.

If cellular Factor XIIa has been separated from other forms of Factor XIIa before the assay is carried out, an assay that does not discriminate between cellular Factor XIIa and other forms of Factor XIIa may be used i.e. a “general” Factor XIIa assay. It may, however, be advantageous to use an assay that is capable of detecting or determining cellular Factor XIIa preferentially in relation to other forms even after a prior separation step.

If no separation step is carried out, the assay used should be capable of detecting or determining the Cellular Factor XIIa under investigation. Various assays for Factor XIIa are described below.

The presence of cellular Factor XIIa, in a tissue sample may be detected using an immunohistological technique. For example, a monoclonal antibody as described below that is labeled with an appropriate label, for example, a fluorescent label, may be used.

In some cases, Factor XII may be measured, rather than Factor XIIa.

Lipid Bound Factor XIIa

The present invention provides a method which comprises detecting or determining lipid bound Factor XIIa in a sample comprising tissue or, especially, a body fluid obtained from a mammalian subject, generally a human.

Measurement of lipid bound Factor XIIa may be performed on a sample of a body fluid, for example, whole blood or plasma. Alternatively, a lipid faction can be isolated from a body fluid or tissue and the Factor XIIa content of the lipid faction determined. A lipid fraction may be isolated as described above under “Separation of forms of Factor XIIa”. For example, lipoproteins may be isolated from a tissue or body fluid, for example, from plasma, for example by precipitation. Suitable agents for precipitating lipoproteins are known and include, for example, reagents comprising sodium chloride, manganese chloride and heparin, and phosphotungstate reagents. Various reagents and methods are described in Demacker, P. N. M. et al. Clinical Chemistry Vol. 43, No. 4, 1997, p 663-668 and in Sharma, A. et al. Clinical Chemistry, Vol. 36, No. 3, 1990, p 529-532.

A sample, for example, plasma, may be centrifuged to remove cellular components, for example, at medium to high speed, for example, at 12,000 to 16,000 g. Lipoproteins may be precipitated using a known lipoprotein precipitation agent, for example, a reagent comprising sodium chloride, manganese chloride and heparin, for example, about 500 mN sodium chloride, about 215 mM manganese dichloride and about 500 U/ml heparin, or using a phosphotungstate precipitation agent, for example, comprising about 50 mM phosphotungstate and generally magnesium chloride.

A resulting precipitate may be isolated, for example, by centrifugation. If desired, a precipitate may be resuspended in the precipitation agent and again isolated. This procedure may be repeated, if desired, for example, two or three times. Washing may be carried out between precipitation steps.

If the lipid bound Factor XIIa has been separated from other forms of Factor XIIa before the assay is carried out, an assay that does not discriminate between different forms of Factor XIIa may be used i.e. a “general” Factor XIIa assay. It may, however, be advantageous to use an assay that is capable of detecting or determining the lipid bound Factor XIIa preferentially in relation to other forms even after a prior separation step.

If no separation step is carried out, the assay used must be capable of detecting or determining the lipid bound Factor XIIa.

In the case of an immunoassay, the lipoprotein fraction may be isolated before or after the sample is contacted with an antibody. It may be advantageous to isolate the lipoprotein fraction after contact with the antibody.

Molecular Complexes and Associations of Factor XIIa with Other Molecular Species

Samples comprising molecular complexes and associations of Factor XIIa with other molecular species, generally samples of body fluids, may be prepared for an assay according to normal practice, see above.

If desired, molecular complexes comprising two or more molecules of Factor XIIa or forms of Factor XIIa in association with low or high affinity binding partners may be separated as described above under “Separation of forms of Factor XIIa” before carrying out an assay for Factor XIIa. For example, Factor αXIIa bound to low affinity binding-partners, Factor βXIIa bound to low affinity binding partners, fragments of Factor βXIIa bound to low affinity binding partners, Factor αXIIa bound to high affinity binding partners, Factor βXIIa bound to high affinity binding partners, and fragments of Factor βXIIa bound to high affinity binding partners, may be separated.

If molecular complexes comprising two or more molecules of Factor XIIa or forms of Factor XIIa in association with low or high affinity binding partners have been separated from other forms of Factor XIIa before the assay is carried out, an assay that does not discriminate between such forms of Factor XIIa and other forms of Factor XIIa may be used i.e. a “general” Factor XIIa assay. It may, however, be advantageous to use an assay that is capable of detecting or determining such forms of Factor XIIa preferentially in relation to other forms even after a prior separation step.

An assay that is capable of detecting or determining a form or forms of Factor XIIa under investigation in preference to other forms may be used without prior separation of the form or forms of Factor XIIa under investigation.

Suitable assays, in particular, immunoassays, are described below.

Immunoassays

An immunoassay may be used according to the present invention to detect or determine one or more forms of Factor XIIa in preference to other forms. An immunoassay may be used in relation to any sample according to the invention.

General Immunoassay Techniques

Methods of carrying out immunoassays are well known, see for example, Tietz Textbook of Clinical Chemistry 2^{nd} Edition” Eds. Burtis, C. A. & Ashwood, E. R., Saunders (1994); Methods in Enzymology, H. Van Vunakis and J. J. Langone (Eds), 1981, 72(B); Practice and Theory of Enzyme Immunoassays, P Tijssen, Laboratory Techniques in Biochemistry and Molecular Biology, R. J. Burden and P. H. Van Knippenberg (Eds), Elsevier, 1985; Introduction to Radioimmunoassay and Related Techniques, T. Chard, ibid, 3rd Edition, 1987; and Methods in Enzymology, H. Van Vunakis and J. J. Langone (Eds) 1981, 74(C).

Immunoassay techniques, both qualitative and quantitative, include ELISA (enzyme linked immunosorbent assays), Western blotting, fluid phase precipitation assays, coated particle assays, competitive assays, sandwich assays, including forward, reverse and simultaneous sandwich assays, and solid phase radio immunoassays (SPRIA).

An antigen-antibody complex may be detected directly, for example, by the techniques described below, or by means of a labeled antibody.

Double Antibody Sandwich Assay

An example of an ELISA format that may be used according to the present invention, is a so-called “double antibody sandwich” assay, in which an antibody, especially a monoclonal antibody, that is capable of binding to one or more forms of Factor XIIa, is immobilized on a solid phase support, for example, on a plastics or other polymeric material, for example on the wells of plastics microtitre plates, or on beads or particles, for example, as used in proprietary systems, for example, the IMx system of Abbott Laboratories, Abbott Park, Ill., USA. This antibody is called a “capture antibody”. A samples is incubated in contact with the immobilised capture antibody. Any form of Factor XIIa that is capable of binding to the immobilized antibody will be “captured” by the immobilized antibody and hence itself immobilized on the solid phase. Factor XIIa that is captured on the solid phase is detected using a labeled antibody that is capable of binding to one or more form of Factor XIIa. This labeled antibody is often called an antibody “conjugate”. By careful selection of the antibodies and/or of other assay conditions, it is possible to optimize the assay such that it preferentially measures, detects and/or determines one or more particular forms of Factor XIIa over other forms.

Labelled Antibodies

A labelled antibody used to detection or detection and/or determination of a target antigen may be polyclonal or monoclonal. Anti-human antibodies, for example, anti-human polyclonal antibodies, are often convenient for use as labelled antibodies for clinical applications. Alternatively, an antibody that binds to the form of Factor XIIa under investigation may be used. Such an antibody may bind, for example, to the heavy chain of Factor αXIIa, to Factor βXIIa, or to a fragment of Factor βXIIa.

The label may be detectable directly or indirectly. Any appropriate radioisotope may be used as a directly detectable label, for example a β-emitter or an γ-emitter, examples being ¹²⁵I, ¹¹³I, ³H, and ²⁴C. For commercial use, non-radioactive labels, generally enzyme labels, are preferred. Enzyme labels are detectable indirectly. An enzyme label is, for example, alkaline phosphatase or a peroxidase, for example, horse radish peroxidase. An appropriate substrate for the chosen enzyme, for example, a substrate that gives rise to a detectable optical or fluorescence change, for example, phenolphthalein monophosphate or a fluorescent substrate, for example, 4-methylumbelliferyl phosphate, is used. Alternatively, there may be used an enzyme reaction that can be followed using an electrochemical method.

A labeled antibody may be used to detect an antigen-antibody complex in, for example, an ELISA, or may form a complex with an antigen, which complex may then be detected. Flow cytometry may be used for detection.

Competitive Assays

One or more forms of Factor XIIa that have been labeled, for example, radiolabelled or enzyme-labelled, may be used in a competitive assay for measurement of one or more forms of Factor XIIa.

Immunoassay for Factor XIIa

An example of an immunoassay for Factor XIIa is the capture assay that described in WO90/08835. To detect or determine one or more forms of Factor XIIa preferentially, it is recommended that mAb 2/215 or an analogue thereof is used, especially as the capture antibody. A different antibody, for example, a polyclonal antibody or a different monoclonal antibody may be used for detection and/or determination of the form of Factor XIIa under investigation, or the same antibody may be used. Selection and/or manipulation of the antibodies and/or the assay conditions enables preferential detection and/or determination of detection of one or more forms of Factor XIIa over other forms.

Further Immunoassay Techniques

Further immunoassay methods for detecting or determining antigens utilise direct detection of a resulting antibody-antigen complex. Examples of such techniques are Surface Plasmon Resonance, Surface Acoustic Wave and Quartz Crystal Microbalance methodologies (Suzuki M, Ozawa F, Sugimoto W, Aso S. Anal Bioanal Chem 372:301-4, 2002; Pearson J E, Kane J W, Petraki-Kallioti I, Gill A, Vadgama P. J Immunol Methods; 221:87-94, 1998; Weisch W, Klein C, von Schickfus M, Hunklinger S. Anal Chem 1996 68:2000-4, 1996; Chou SF, Hsu W L, Hwang J M, Chen CY. Clin Chem 48:913-8, 2002).

If a labelled antibody forms a complex with an antigen the complex may be detected or determined by flow cytometry.

Standards and Controls

Immunoassays generally use “standards” as reference points.

A standard suitable for an assay for detection or detection and/or determination of one or more forms of Factor XIIa may typically comprise a solution containing known amounts of one or more appropriate forms of Factor XIIa. Alternatively, a standard may comprise one or more appropriate forms of Factor XIIa bound to a supporting material such as a solid phase.

The materials used act as standards and controls may take various forms dependent upon the assay to be used. In some assay formats, suitable material may be in aqueous solution. Factor XII or a fragment thereof, including the various forms of Factor XIIa. In other formats, for example where the same antibody is used as the capture and detection (conjugate) antibody in an ELISA, it may be desirable to create constructs containing multiple Factor XII molecules or fragments thereof, including the various forms of Factor XIIa, for example, by binding Factor βXIIa to the surface of beads, for example, polycarbonate beads, for example, 3 μM in diameter.

A standard suitable for an assay for detection or detection and/or determination of lipid bound Factor XIIa typically comprises a solution containing known amounts of lipid bound Factor XIIa. Alternatively, a standard may comprise Factor XIIa bound to a non-lipid supporting material, for example, a solid phase, or an aqueous solution of Factor XIIa may be used as a standard.

A standard suitable for an assay for detection or detection and/or determination of urinary Factor XIIa would typically comprise of a solution containing a known amount of Factor XIIa.

Immunohistology

The presence of a form or forms of Factor XIIa in a tissue sample may be detected using an immunohistological technique. For example, a monoclonal antibody as described above, labeled with an appropriate label, for example, a fluorescent label, may be used. Typically a labeled antibody is contacted and incubated with a tissue sample, the reagents are subsequently washed off under conditions that do not disrupt any antibody-antigen complexes that have formed, and any such complexes are detected.

Chromogenic Assays

Detection or determination of one or more forms of Factor XIIa may be performed by measuring its enzyme activity using a chromogenic substrate for example, as described by Vinazzer H., Thromb Res., 14, 155-66, 1979.

This assay may involve a step where one or more forms of Factor XIIa are isolated from other forms, see above.

Immunoassay for Cellular Factor XIIa

Cells may be isolated from a body fluid, for example, from blood or plasma, for example, by centrifugation and washing, preferably at least once and especially two or more times, for example, in a suitable medium that does not affect the cellular Factor XIIa, for example, that does not cause cellular Factor XIIa to become dissociated from cells. Suitable liquids are generally buffers, for example, phosphate buffered saline (PBS), for example, at pH 7.4.

A sample of a body fluid comprising cells may be washed, centrifuged at high speed, and then suspended in a suitable liquid give “washed cells”. An example of high speed centrifugation is 16,000 g for 10 minutes. An example of a suitable washing and suspending liquid is PBS pH 7.4. One or more, for example, two or three, or more, rounds of centrifugation may be carried out.

Cell rich plasma may be obtained, for example, by low speed centrifugation of blood, for example, by centrifuging citrated blood from 10 minutes at 1000 g. Further centrifugation, for example, high speed centrifugation, of cell rich plasma, for example, centrifugation at 16,000 g for 10 minutes, gives a supernatant, called cell poor plasma.

Using mAb 2/215 or an analogue thereof as both capture and detection antibody for a double antibody sandwich assay with cell rich, cell poor and wshed cells as samples, the maximal response was obtained for the washed cells, with a minimal response for the cell poor plasma. In contrast, when a polyclonal antibody was used as the detection antibody with mAb 2/215 or an analogue thereof as the capture antibody, significant responses were obtained for both the cell rich and cell poor plasmas, but only a minimal response for the washed cells.

Further immunoassay and flow cytometry experiments confirmed the results. These results indicate that mAb 2/215 binds to epitope(s) on Factor XIIa that are available when the Factor XIIa is cellular, whereas the epitope(s) on Factor XIIa to which mAb 201/9 bind are less available for binding when the Factor XIIa is present on the surface of the cell.

Immunoassay for Lipid Bound Factor XIIa

An immunoassay may be carried out using mAb 2/215 or an analogue thereof or a fragment thereof, for example, a Fab fragment. In the case of a capture assay, it is preferably to use mAb 2/215 or an analogue thereof as the capture antibody. A different antibody, for example, a polyclonal antibody or a different monoclonal antibody, or the same antibody may be used for detection.

A direct immunoassay, for example, a radioimmunoassay, may be used. In such a case it is preferable to use mAb 2/215 or an analogue thereof or a fragment thereof, for example, a Fab fragment. Examples of suitable labels are given above.

The lipoprotein fraction may be isolated before or after the sample is contacted with an antibody. It may be advantageous to isolate the lipoprotein fraction after contact with the antibody. The lipoprotein fraction may be isolated as described above in the “Sample preparation” section.

As an alternative to an immunoassay, detection and/or determination of lipid bound Factor XIIa may be performed by measuring its enzyme activity using a chromogenic substrate for example, as described by Vinazzer H., Thromb Res., 14, 155-66, 1979. This may involve a stage where one or more species are isolated from other species, for example, as described above.

Immunoassay for Molecular Complexes and Associations of Factor XIIa with Other Molecular Species

An immunoassay may be carried after separation of molecular complexes and associations of Factor XIIa with other molecular species form other forms of Factor XIIa, or on a sample without such separation. For example, if desired, molecular complexes comprising two or more molecules of Factor XIIa or forms of Factor XIIa in association with low or high affinity binding partners may be separated as described above under “Separation of forms of Factor XIIa” before carrying out an assay for Factor XIIa. For example, Factor αXIIa bound to low affinity binding partners, Factor βXIIa bound to low affinity binding partners, fragments of Factor βXIIa bound to low affinity binding partners, Factor αXIIa bound to high affinity binding partners, Factor βXIIa bound to high affinity binding partners, and fragments of Factor βXIIa bound to high affinity binding partners, may be separated.

Any of the immunoassays described above may be used to determine molecular complexes and associations of Factor XIIa with other molecular species. As described above, it is often preferable to use mAb 2/215 or an analogue thereof as an antibody, in particular as the capture antibody in a capture immunoassay. The labelled antibody used for detection should be capable of binding to the captured form of Factor XIIa. For example, the labelled antibody may bind to the heavy chain of Factor αXIIa, to Factor βXIIa, or to Factor βXIIa fragments.

Immunoassay and Other Assays for Urinary Factor XIIa

Any of the immunoassays described above may be used to determine one or more forms of Factor XIIa in urine preferentially relative to other forms. As described above, it is generally preferable to use mAb 2/215 or an analogue thereof is used as an antibody, in particular as the capture antibody in a capture immunoassay.

Kits

The present invention further provides a kit for carrying out an immunoassay of the present invention, which kit comprises, each in a separate container or otherwise compartmentalised: (i) a monoclonal antibody that is capable of binding to one or more forms of Factor XIIa, for example, mAb 2/215 or an analogue thereof or another monoclonal antibody having the same or similar Factor XIIa binding properties as mAb 2/215 or an analogue thereof, and (ii) a labeled antibody capable of binding to one or more forms of Factor XIIa when one or more forms of Factor XIIa is bound to the monoclonal antibody defined in (i).

The kit may comprise further components for carrying out an immunoassay, for example, as described above. The monoclonal antibody may be immobilised on a solid support.

A kit according to the invention may comprise, for example,
a) a monoclonal antibody that is capable of binding to one or more forms of Factor XIIa, for example, mAb 2/215 or an analogue thereof or another monoclonal antibody having the same or similar Factor XIIa binding properties as mAb 2/215 or an analogue thereof,
(b) a standard typically comprising of a solution containing known amounts of one or more forms of Factor XIIa
(c) labelled antibody capable of reacting with one or more forms of Factor XIIa when one or more forms of Factor XIIa is bound to the monoclonal antibody defined in (i).

The materials used act as standards and controls may take various forms dependent upon the assay to be used. In some assay formats, suitable material may be in aqueous solution. Factor XII or a fragment thereof, including the various forms of Factor XIIa. In other formats, for example where the same antibody is used as the capture and detection (conjugate) antibody in an ELISA, it may be desirable to create constructs containing multiple Factor XII molecules or fragments thereof, including the various forms of Factor XIIa, for example, by binding Factor βXIIa to the surface of beads, for example, polycarbonate beads, for example, 3 μM in diameter.

Further examples of standards are given above. Alternatively, a kit may comprise labeled forms of Factor XIIa, for use in a competitive assay.

A kit may also comprise further components, each in a separate container, for example, diluent(s), wash reagent solution(s) and substrate solution(s).

Assay Devices

The present invention also provides an assay device suitable for carrying out an assay of the invention. The term “assay device” is used herein to denote means for carrying out an immunoassay comprising a solid phase, generally a laminar solid phase, for example, a membrane, sheet, strip, coating, film or other laminar means, on which is immobilized an appropriate capture antibody. The immobilized antibody is preferably present in a defined zone, called herein the “antigen capture zone”.

An assay device may incorporate the solid phase within a rigid support or a housing, which may also comprise some or all of the reagents required for carrying out an assay. Sample is generally applied to an assay device at a predetermined sample application zone, for example, by pouring or dripping the sample on the zone, or by dipping the relevant part of the device into the sample. If the sample application zone is at a different site from the antibody capture zone, the arrangement of the device is generally such that antigens in the sample migrate to the antibody capture zone. The required reagents are then applied in the appropriate order at designated application zones, which may or may not be the same as the sample application zone. Again, if the or any reagent application zone is at a different site from the antibody capture zone, the arrangement of a device is generally such that the reagent(s) migrate to the antibody capture zone, where any antigen-antibody complex formed is detected. All or some of the reagents required for an immunoassay may be incorporated within a device, in liquid or dry form. If so, a device is generally arranged such that interactions between different parts of the device, which interactions may occur automatically during the operation of the device or may be brought about by the user of the device, bring the various reagents into contact with one another in the correct sequence for the immunoassay to be carried out.

A wide variety of assay devices are described in the literature of immunoassays. Examples of membrane devices are described in U.S. Pat. Nos. 4,623,461 and 4,693,984. Depending on their design and their speed of action, some assay devices are called “dipsticks” and some are called “rapid assay” devices. A “rapid assay” device generally provides a result within ten minutes of the application of sample. (A typical microtitre plate or bead assay requires incubation steps, and generally takes at least an hour to provide a result.) Accordingly, although assay devices are generally more expensive than microtitre or bead format assays, they have particular uses in clinical testing, for example, when a result is required rapidly, for example, in the case of emergency treatment.

Assay devices have the particular advantage that they can be used without the need for sophisticated laboratory facilities or even without the need for any laboratory facilities. They may therefore be used for “Point of Care” testing, for example, in an emergency room, in a doctor's surgery, in a pharmacy or, in certain cases, for home testing. They are particularly useful in territories where laboratory facilities are few and far between.

Monoclonal Antibodies

The present invention relates to the use of a monoclonal antibody that is capable of binding to one or more particular forms of activated Factor XII in preference to other forms, for example, mAb 2/215 and monoclonal antibodies having the same or similar properties as mAb 2/215 in relation to binding to Factor XIIa.

As stated above, it appears that, when Factor XIIa is cellular, that is to say, bound to cells or cellular material, not all Factor XIIa epitopes appear to be as accessible as when Factor XIIa is not cellular. For example, monoclonal antibody 2/215 is capable of binding effectively to cellular Factor XIIa and to non-cellular Factor XIIa. However, monoclonal antibody 201/9 and a sheep polyclonal antibody raised against Factor βXIIa do not appear to be able to bind as effectively to cellular Factor XIIa as to non-cellular Factor XIIa.

Without being bound by the following hypothesis, it appears that mAb 2/215 is able to bind effectively to epitopes that are available when Factor XIIa is in the form of a complex or an association, for example, with cellular material, with lipids, with one or more other molecules of Factor XIIa, or with a low or high affinity binding partner.

Monoclonal antibodies that have binding characteristics that are the same as or similar to those of mAb 2/215 and mAb 201/9 in relation to binding to particular forms of Factor XIIa may be produced by conventional methods and screened by conventional methods for the desired binding characteristics. For example, a monoclonal antibody that is capable of binding preferentially to one or more particular forms of Factor XIIa, for example, a monoclonal antibody having the same or similar properties as mAb 2/215 in relation to binding to Factor XIIa, may be produced by methods that are known per se. Resulting antibodies may be screened for those having the desired characteristics.

It is generally preferable that a monoclonal antibody for use according to the present invention shows no significant binding to Factor XII zymogen. The corrected cross-reactivity with Factor XII is, for example, 0.1% or less. A factor to take into consideration in assessing the cross-reactivity of an antibody of the invention with Factor XII is that even “pure” Factor XII preparations are almost inevitably contaminated with small amounts of Factor XIIa (Silverberg and Kaplan, Blood 60, 1982, 64-70). WO90/08835 gives details of methods of assessing the corrected cross-reactivity with Factor XII. Unless specified otherwise, the term “cross reactivity” is used herein to mean the corrected cross reactivity.

Methods used to produce monoclonal antibodies are well known, see for example, Methods in Enzymology, H. Van Vunakis and J. J. Longone (Eds) 1981, 72(B) and ibid, 1983 92(E). Monoclonal antibodies may be produced, for example, by a modification of the method of Kohler and Milstein (G. Kohler and C. Milstein, Nature, 1975, 256, 495).

WO90/08835, which is incorporated herein by reference, describes in general terms how to produce an antibody that binds to Factor αXIIa and to Factor βXIIa and that has shows a corrected cross-reactivity with Factor XII of 0.1% or less, and gives specific details of the production of mAb 2/215 and mAb 201/9. The general and specific methods described therein may used to produce a monoclonal antibody suitable for use according to the present invention, for example, a monoclonal aneibody having the same or similar properties in relation to binding to Factor XIIa as mAb 2/215 or as mAb 201/9. A general protocol for producing monoclonal antibodies suitable for use according to the present invention, based on the disclosure of WO90/08835, is given in Example 22 below.

Methods used to produce monoclonal antibodies are well known, see for example, Methods in Enzymology, H. Van Vunakis and J. J. Longone (Eds) 1981, 72(B) and ibid, 1983 92(E). Monoclonal antibodies may be produced, for example, by a modification of the method of Kohler and Milstein (G. Kohler and C. Milstein, Nature, 1975, 256, 495). The immunogen used in the production of monoclonal antibodies may be Factor βXIIa, see WO90/08835. Resulting monoclonal antibodies may be screened for those that show no significant binding to Factor XII zymogen, for example, having a corrected cross-reactivity with Factor XII of 0.1% or less.

Resulting monoclonal antibodies may be screened for binding to the form of Factor XIIa to which binding is desired, for example, cellular Factor XIIa, lipid bound, Factor XIIa or a complex or association of Factor XIIa with other Factor XIIa molecules or with high or low binding affinity partners.

It may be advantageous to use monoclonal antibody 2/215 or 201/9, respectively, as a reference antibody in screening for antibodies that bind to specific forms of Factor XIIa. A selected antibody may have binding characteristics for selected forms of Factor XIIa that are the same as or similar to those of mAb 2/215 or 201/9, respectively.

Although the hybridoma used to produce mAb 2/215 was derived from mouse spleen cells, the invention is not limited to hybridomas of murine or part-murine origin. Both fusion partners (spleen cells and myelomas) may be obtained from any suitable animal. Recombinant antibodies may be produced. Antibodies may be brought into chimeric or humanized form, if desired. Hybridomas are preferably cultured in vitro.

Polyclonal Antibodies

The present invention also provides polyclonal antibodies, also called a polyclonal antiserum, that are capable of reacting selectively with one or more forms of Factor XIIa. Such antibodies may be labeled and used for detection of one or more captured forms of Factor XIIa, in an ELISA.

The invention also provides a method for the production of such a polyclonal antiserum, which comprises administering Factor XIIa, for example, Factor βXIIa to an animal, obtaining serum from the animal, screening the serum for binding to one or more forms of Factor XIIa. IN some cases, Factor XII can be used as the immunogen.

The invention also includes a method which comprises detecting or determining Factor XIIa in a sample comprising urine obtained from a subject. In this embodiment of the invention it is not necessary to detect or determine any one of more forms of Factor XIIa preferentially in relation to other forms. An assay that does not discriminate between forms may be used. Such an assay may be, for example, a chromogenic assay or an immunoassay. An immunoassay may be, for example, as described in WO90/08835.

Assay of Factor XIIa in urine, whether by means of a “general” assay or an assay that can discriminate between different forms of Factor XIIa, provides useful information in relation to renal function, renal disease and renal damage, as Factor XIIa concentrations in urine are a sensitive marker of renal function, renal disease and renal damage, particular in conditions where extensive proteinuria is not present. Elevated concentrations of Factor XIIa in urine of a subject, for example, relative to healthy subjects, is indicative of any one of impaired renal function, renal disease and renal damage. Changes in the concentration of urinary Factor XIIa may be indictative of change in in a clinical condition, for example, exacerbation of the condition or improvement, for example, in response to therapy.

Clinical and Other Utility

The invention, especially the immunoassays described above, provides a method of detection and/or determination of different forms of Factor XIIa that can be used readily on automated equipment for large scale use.

Factor XII and its activated form, Factor XIIa, are considered to be involved in blood coagulation and other contact systems, also known as contact phase systems, for example, fibrinolysis, complement cascade, inflammation and vaso-dilation, see Jacobsen S. and Kriz M., Br J. Pharmacol., 29, 25-36, 1967; Kurachi K et al, Biochemistry, 19, 1330-8 1980; Radcliffe R et al, Blood, 50, 611-7, 1977; Ghebrehiwet B et al, J Clin Invest, 71, 1450-6. 1983; Z Toossi et al, Proc Natl Acad Sci USA, 89, 11969-72, 1992; Wachtfogel Y T et al, Blood 67, 1731-7, 1986; Wachtfogel Y T et al, Thromb Haemost, 80, 686-91, 1998; and Schreiber et al AD, J Clin Invest., 52, 1402-9, 1973.

As Factor XII and its activated form, Factor XIIa are involved in haemocoagulation and have a role in maintaining vascular wholeness and blood pressure, in influencing various functions of endothelial cells, in control of fibrinolysis and in maintaining the constitutive anticoagulant character of the intravascular space, measurement of specific forms of Factor XIIa is useful in investigations of those systems, including for example, fibrinolysis, complement cascade, and vasodilation, and also in investigations relating to thrombosis and stenosis.

Clinical and experimental studies indicate that the contact system, which includes Factor XIIa, is involved in acute and chronic inflammation, shock of different aetiologies including septic shock, diabetes, allergy, thrombo-haemorrhagic disorders including disseminated intravascular blood coagulation, oncological diseases, cardiovascular conditions, for example, myocardial infarction, angina and acute coronary syndrome, angiogenesis, sepsis, spontaneous abortion and thromboembolism.

The involvement of Factor XIIa in haemocoagulation, in maintaining vascular wholeness and blood pressure, in control of fibrinolysis and in maintaining the constitutive anticoagulant character of the intravascular space supports the clinical and experimental observations of the involvement of Factor XIIa in thrombo-haemorrhagic disorders including disseminated intravascular blood coagulation, oncological diseases, cardiovascular conditions, for example, myocardial infarction, angina and acute coronary syndrome, angiogenesis, and thromboembolism.

Our surprising observation that Factor XIIa is present on granulocytes, which are activated/involved in the inflammatory process, supports the clinical and experimental studies that implicate Factor XIIa in various conditions that involve inflammation, for example, acute and chronic inflammation, shock of different aetiologies including septic shock, allergy, oncological diseases, and sepsis.

Detection and/or determination of specific forms of Factor XIIa, are therefore useful in clinical and scientific investigations of diseases and disorders in which the contact system may be involved, including diagnosing, monitoring, or predicting the susceptibility to, progress of, or outcome of such a disease or disorder, or of treatment of the disease or disorder in a subject having or suspected of having the disease or disorder. Such diseases and disorders include acute and chronic inflammation, shock of different aetiologies, diabetes, allergy, thrombo-haemorrhagic disorders including disseminated intravascular blood coagulation and thromboembolism, thrombosis and stenosis, oncological diseases, cardiovascular conditions, for example, myocardial infarction, angina, acute coronary syndrome, angiogenesis, sepsis, and spontaneous abortion.

Detection or determination of one or more forms of Factor XIIa, is therefore useful as an aid to diagnosing, monitoring, or predicting the susceptibility to, progress of, or outcome of a disease or disorder, or of treatment of the disease or disorder in a subject having or suspected of having the disease or disorder, in which disease or disorder the amount of one or more forms of Factor XIIa is different from that in healthy subjects. Changes in the concentration of one or more forms of Factor XIIa may be indicative of any of the diseases and disorders mentioned above. Changes in concentration in a subject with time may be indicative of change in the condition, for example, exacerbation of the condition, or improvement, for example, in response to therapy. Such methods of diagnosing, monitoring, predicting the susceptibility to, progress of, or outcome of a disease or disorder, or of treatment of the disease or disorder, called “diagnosis, prediction and monitoring”, are part of the present invention.

In addition, Factor XIIa in urine is a sensitive marker of renal function, renal disease and renal damage, and detection or determination of Factor XIIa in urine can provide useful information on renal function, renal disease and renal damage.

Diagnosis, Prediction and Monitoring

The present invention provides a method for diagnosing, monitoring, or predicting the susceptibility to, progress of, or outcome of a disease or disorder, or of treatment of the disease or disorder in a subject having or suspected of having the disease or disorder, which comprises detecting or determining one or more forms of Factor XIIa in preference to other forms of Factor XIIa in a sample obtained from the subject, and comparing the results obtained for the subject with the results obtained using the same assay for samples obtained from at least any one or more of the following:
(i) subjects having the disease or disorder;
(ii) subjects having the disease or disorder, which subjects were monitored in relation to the progress and/or outcome of the disease or disorder;
(iii) subjects having the disease or disorder and the treatment;
(iv) subjects having the disease or disorder and the treatment, which subjects were monitored in relation to the treatment in relation to the progress and/or outcome of the disease or disorder;
(v) subjects who do not have the disease or disorder;
(vi) the same subject before the onset of the disease or disorder or before the start of the treatment of the disease or disorder; and
(vii) the same subject at an earlier or later stage of the disease or disorder or the treatment of the disease or disorder or before the onset of the disease or disorder.

The sample may be any of those described above. For example, the sample may be a sample of a body fluid, for example, blood, plasma, serum, urine, cerebrospinal fluid, saliva, or tears.

The assay may be for the detection and/or detection and/or determination of one or more forms of Factor XIIa, for example, any one or more selected forms, for example, any one or more of cellular Factor XIIa, lipid bound Factor XIIa and urinary Factor XIIa.

Specificity of an assay for one or more forms of Factor XII over other forms may be achieved or improved by design of the assay, as described above. In the case of an immunoassay, such design may include any one or more of choice of the antibody or combination of antibodies to be used; presence, absence and choice of a detergent; and conditions used for plate coating in the case of an antigen capture assay involving an antibody coated on a solid phase, see above.

The assay for Factor XIIa may be an immunoassay that comprises the use of an antibody that is capable of binding to the form or forms of Factor XIIa under investigation. In such an assay an antibody that is capable of binding to the form or forms of Factor XIIa under investigation is immobilized on a solid phase as a capture antibody.

Alternatively or in addition, an antibody that is capable of binding to the form or forms of Factor XIIa under investigation is labeled with a label that is detectable directly or indirectly.

In an immunoassay, wherein a resulting antibody-antigen complex may be determined directly, for example, as described above in the section “Immunoassays”.

In an immunoassay an antibody that is capable of binding to the form or forms of Factor XIIa under investigation may be mAb 2/215 or an analogue thereof, mAb 201/9 or an analogue thereof, or a polyclonal antibody that is capable of binding to Factor XIIa and optionally also to Factor XII.

In an immunoassay in which mAb 2/215 or an analogue thereof, mAb 201/9 or an analogue thereof, or a polyclonal antibody that is capable of binding to Factor XIIa is used, the antibody may be labeled with a label that is detectable directly or indirectly and/or may be immobilized on a solid phase as a capture antibody.

Any Factor XIIa captured by the defined antibody may be detected or determined using a labeled antibody, for example, as defined above.

The disease or disorder under investigation may be any of those described above in the “Clinical utility” section, for example, diseases and disorders of the coagulation system; conditions that involve hemaocoagulation, fibrinolysis, kininogensis, complement activation or angiogenesis, maintaining vascular wholeness and blood pressure, maintaining the constitutive anticoagulant character of the intravascular space, or tissue defence and repair; conditions that involve acute or chronic inflammation, shock of any aetiology, diabetes, allergy, a thrombo-haemorrhagic disorder, sepsis, spontaneous abortion or an oncological disease; and conditions that involve intravascular blood coagulation or thromboembolism, thrombosis or stenosis, myocardial infarction, acute coronary syndrome or angina.

Treatment of the clinical or pathological condition may involve administration of a therapeutic agent and/or may involve a surgical procedure. For example, treatment of thrombosis or stenosis may involve coronary artery angioplasty and/or thrombolysis.

It may be advantageous to test a series of samples obtained from a subject, for example, samples obtained during the course the disease or disorder and/or samples obtained during treatment of the disease or disorder and/or before treatment is started.

The disease or disorder may be or involve thrombosis or stenosis and/or treatment may involve coronary artery angioplasty or thrombolysis. In one embodiment of the invention, for diagnosing, monitoring, or predicting the progress or outcome of such conditions and treatment, the immunoassay used may be a capture assay in which the capture antibody is mAb 2/215 or an analogue thereof, the labeled antibody is a polyclonal anti-Factor XIIa antibody, and no detergent (Triton) is present in the sample during the sample (first) incubation step. It has been shown (see Example 16) that when concentrations of Factor XIIa measured before and after the angioplasty or thromoblytic procedure, increased concentrations of Factor XIIa after the procedure indicate efficacy of the procedure. It is considered that the assay may measure molecular complexes comprising two or more molecules of Factor XIIa and/or particles comprising multiple Factor XIIa molecules.

The disease or disorder may be suspected myocardial infarction or acute coronary syndrome. Assays for different forms of Factor XIIa give different information regarding the prediction of the progress or outcome of such conditions.

In one embodiment of the invention the immunoassay used is a capture assay in which the capture antibody is mAb 2/215 or an analogue thereof, the labeled antibody is mAb 2/215 or an analogue thereof and detergent (Triton) is present in the sample during the sample (first) incubation step. It has been shown that when the samples are obtained on admission to hospital, low concentrations of Factor XIIa obtained using the 2/215 capture antibody 2/215 labeled antibody assay are associated with an increased risk of a secondary troponin positive event within the initial hospitalization period, (see Example 17).

In another embodiment of the invention the immunoassay used is a capture assay in which the capture antibody is mAb 2/215 or an analogue thereof, and the labeled antibody is mAb 201/9, and detergent (Triton) is present in the sample during the sample (first) incubation step, this assay detects particular forms of Factor XIIa preferentially to others. It has been shown that when the samples are obtained on initial admission to hospital increased concentrations of Factor XIIa obtained using the 2/215 capture antibody 201/9 labeled antibody assay are indicative of an increased risk of a secondary troponin positive event within 30 days of the date of admission to hospital, (see Example 17).

In a further embodiment of the invention the immunoassay used is a capture assay in which the capture antibody is mAb 2/215 or an analogue thereof, the labeled antibody is a polyclonal anti-Factor XII antibody, and no detergent (Triton) is present in the sample during the sample (first) incubation step, this assay detects particular forms of Factor XIIa preferentially to others. It has been shown that when the samples are obtained on initial admission to hospital, a high or a low concentration of Factor XIIa relative to intermediary concentrations obtained using the 2/215 capture antibody polyclonal anti-Factor XII assay is indicative of a high risk of death (see Example 17). It is considered that the assay may measure molecular complexes comprising two or more molecules of Factor XIIa and/or particles comprising multiple Factor XIIa molecules.

The disease or disorder may be sepsis. In one embodiment of the invention, when samples are analysed using an immunoassay that involves the use of mAb 2/215 or an analogue thereof, it has been shown that increased concentrations of certain forms of Factor XIIa, which were cellular Factor XIIa, are indicative of sepsis. This result is consistent with our surprising observation that Factor XIIa is present on granulocytes, which are activated/involved in the inflammatory process, and supports our hypothesis that Factor XIIa is implicated in various conditions that involve inflammation.

As stated above, Factor XIIa in urine is a sensitive marker of renal function, renal disease and renal damage. The present invention relates to a method for diagnosing or monitoring diseases or disorders in which Factor XIIa, in particular the concentration of Factor XIIa in the urine of a subject having the disease or disorder is different from that in a healthy subject.

The present invention provides a method for diagnosing or monitoring a disease or disorder, or monitoring treatment of the disease or disorder, which comprises detecting or determining Factor XIIa, in particular the concentration of Factor XIIa, in the urine of a subject having or suspected of having the disease or disorder.

For example, the present invention provides a method for diagnosing or monitoring renal function, renal disease or renal damage, or monitoring treatment of impaired renal function, renal disease or renal damage in a subject having or suspected of having impaired renal function, renal disease or renal damage, which comprises detecting or determining Factor XIIa in a sample obtained from the subject.

Generally the results obtained for the subject are compared with the results obtained using the same assay for samples obtained from at least any one or more of the following:
(i) subjects having the disease or disorder, for example, impaired renal function, renal disease or renal damage;
(ii) subjects having the disease or disorder, for example impaired renal function, renal disease or renal damage, which subjects were monitored in relation to the progress and/or outcome of the disease or disorder, for example impaired renal function, renal disease or renal damage;
(iii) subjects having the disease or disorder, for example impaired renal function, renal disease or renal damage and having the treatment therefor;
(iv) subjects having the disease or disorder, for example impaired renal function, renal disease or renal damage and the treatment, which subjects were monitored in relation to the treatment in relation to the progress and/or outcome of the disease or disorder, for example impaired renal function, renal disease or renal damage;
(v) subjects who do not have the disease or disorder, for example impaired renal function, renal disease or renal damage;
(vi) the same subject before the onset of the disease or disorder, for example impaired renal function, renal disease or renal damage or before the start of the treatment of the disease or disorder, for example impaired renal function, renal disease or renal damage; and
(vii) the same subject at an earlier or later stage of the disease or disorder, for example impaired renal function, renal disease or renal damage or the treatment, or before the onset of the disease or disorder, for example impaired renal function, renal disease or renal damage.

The Factor XIIa may be detected or determined by an assay that is capable of detecting or determining one or more of Factor XIIa preferentially relative to other forms, or may be detected or determined by means of an assay that does not discriminate between forms of Factor XIIa.

Determination of Assays that Provide Clinically Useful Information

To practise the present invention, it is not essential to identify which particular form of Factor XIIa is associated with a particular disease or disorder. It is sufficient to know or to determine by means of simple procedures that a particular form of Factor XIIa does have an association with a disease or disorder, for example, with the appearance, progression or outcome of the disease or disorder or, for example with the effectiveness or outcome of treatment of the disease or disorder.

As disclosed above, Factor XIIa has long been known to be involved in the contact system of blood coagulation in vivo. More recent studies indicate that Factor XIIa is also involved in other systems that relate to haemocoagulation and complement activation, and further clinical and experimental results are indicating the contact system is involved in many other conditions. Such conditions are described above in the “Clinical utility” section. It can be established by simple procedures, for example as described below, whether a form of Factor XIIa has a clinically relevant association with a disease or disorder or with treatment of a disease or disorder.

Accordingly, the present invention is not limited to those diseases or disorders that are currently known to have an association with Factor XIIa. It can be determined by means of simple procedures whether a clinically relevant association exists between Factor XIIa and the disease or disorder.

The present invention provides a method comprising carrying out a series of assays for Factor XIIa on samples obtained from subjects having a disease or disorder or treatment for a disease or disorder, and selecting an assay that provides information on Factor XIIa levels that is relevant to the disease or disorder or the treatment.

The present invention also provides a method for providing an assay for Factor XIIa suitable for providing information relevant for diagnosing, monitoring, or predicting the susceptibility to, progress of, or outcome of a disease or disorder, or of treatment of the disease or disorder in a subject having or suspected of having the disease or disorder, which comprises carrying out a series of assays for Factor XIIa on samples obtained from subjects having the disease or disorder or the treatment, and determining which assay(s) provide information on Factor XIIa levels that is relevant to diagnosing, monitoring, or predicting the susceptibility to, progress of, or outcome of the disease or disorder, or of treatment of the disease or disorder.

The method preferably comprises comparing the results obtained for Factor XIIa in the samples obtained from subjects having the disease or disorder otr the treatment with the results obtained using the same assay for samples obtained from at least any one or more of the following:
(i) subjects having the disease or disorder;
(ii) subjects having the disease or disorder, which subjects were monitored in relation to the progress and/or outcome of the disease or disorder;
(iii) subjects having the disease or disorder and the treatment;
(iv) subjects having the disease or disorder and the treatment, which subjects were monitored in relation to the treatment in relation to the progress and/or outcome of the disease or disorder;
(v) subjects who do not have the disease or disorder;
(vi) the same subject before the onset of the disease or disorder or before the start of the treatment of the disease or disorder; and
(vii) the same subject at an earlier or later stage of the disease or disorder or the treatment of the disease or disorder or before the onset of the disease or disorder.

The samples analyzed are preferably a series of samples obtained from the various subjects for example in the course of the disease or disorder, or during the course of the treatment.

The assay used may be any of those described above in relation to the practice of the present invention, including immunoassays and other assays. If a clinically relevant association is found between particular form(s) of Factor XIIa and a disease or disorder or treatment of a disease or disorder using a particular assay, that assay may subsequently be used for diagnosing, monitoring, or predicting the susceptibility to, progress of, or outcome of a disease or disorder, or of treatment of the disease or disorder according to the present invention, and as described above.

It may be useful to assemble results obtained pertaining to the relationship between various assays and various conditions into a database that can subsequently be used to assist the interpretation of results obtained from particular subjects under investigation. Such a method is part of the present invention as is a database comprising results assembled as described above.

As can be seen, the discovery that Factor XIIa exists in different forms, and that there are clinically relevant associations between different forms and diseases and disorders and treatment of diseases and disorders has practical utility that extends beyond the coagulation system which Factor XIIa has traditionally been associated and also beyond those diseases and disorders that have been identified to date as having an association with Factor XIIa

The following non-limiting Examples illustrate the present invention.

### EXAMPLES

### Example 1

In this example the existence of multiple species of Factor XIIa in plasma was demonstrated by binding to fluorescently labelled antibody, and separating the resultant complexes on the basis of molecular weight using high performance liquid chromatography (HPLC).

Antibody 2/215 was labelled with Fluorescein Isothiocyanate (FITC) (Pierce, 3747 N Meridian Road, PO Box 117, Rockford, Ill. 61105) in accordance with the manufacturer's instructions.

The HPLC system consisted of a Waters 1525 Binary HPLC Pump, a Waters 2487 Dual λ (wavelength) Absorbance Detector, and Jasco FP1520 Integral Fluorescence Detector.

The mobile phase used for the HPLC was 0.1M NaCl 0.05M Tris HCl, 0.4% (w/v) Tri-sodium citrate pH 7.5. The stationary phase comprised 2×30 cm BioSep-SEC-S 3000 columns in series (Phenomenex, Queens Avenue, Hurdsfield Industrial Estate, Macclesfield, Cheshire SK10 2BN, United Kingdom). Flow rate was 1.0 ml min⁻¹ and the injection volume was 100 μl. Settings for the Jasco Fluorescence detector were: Excitation wavelength 494 nm, emission wavelength 520 nm, Gain 1000, attenuation 1.

Samples run on the HPLC system were the FITC labelled 2/215 alone, a blood plasma sample alone, and blood plasma which had been incubated with FITC labelled 2/215 for 4 hours (250 μl plasma plus 1 μl FITC labelled antibody).

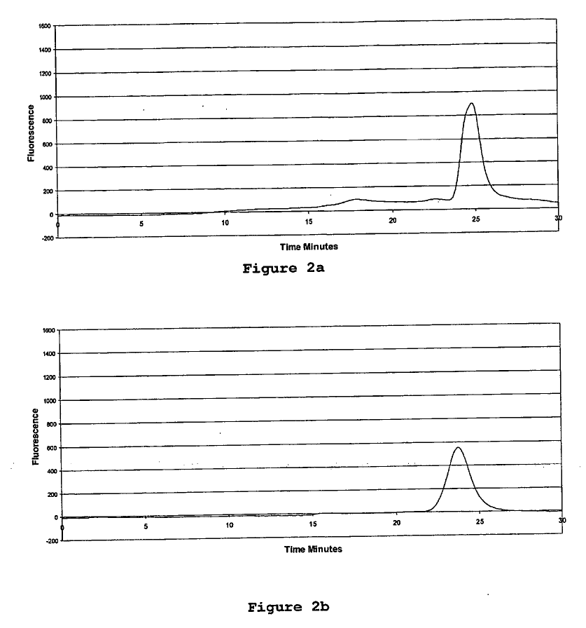

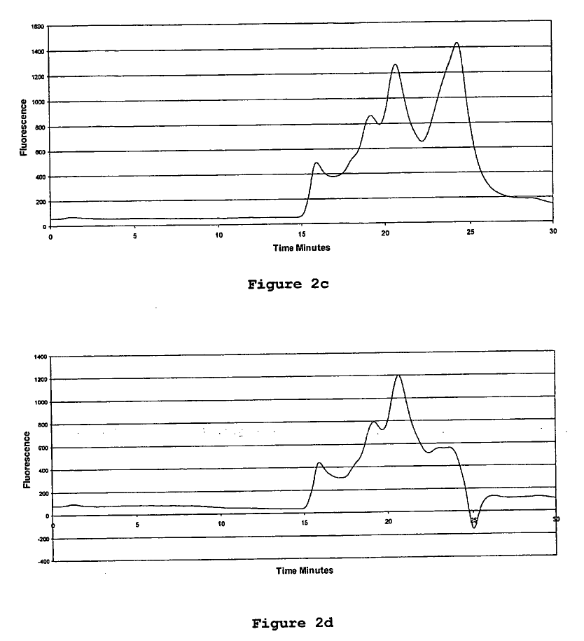

Examples of plots of fluorescence versus time are shown in FIGS. 2a to 2d.

In FIG. 2a, from the trace for the plasma sample alone it can be seen that the plasma sample exhibits endogenous fluorescence. In FIG. 2b, fluorescence associated with the FITC labelled antibody is observed. In FIG. 2c, which relates to plasma which has been preincubated with FITC labelled antibody a number of peaks additional to those in FIGS. 2a and 2b are observed. This indicates that the FITC labelled antibody is binding to several components in the plasma sample. This is further exhibited in the trace shown in FIG. 2d, where the signals associated with endogenous fluorescence and the FITC labelled antibody alone have been subtracted, the resultant trace reflecting only the binding of the antibody to species in plasma.

### Example 2

In this example the existence of multiple species of activated Factor XII in plasma was demonstrated by binding to antibody fragments labelled with a radiotracer (Iodine 125), and separating the resultant complexes on the basis of molecular weight using high performance liquid chromatography (HPLC).

Fab antibody fragments of antibody 2/215 were prepared using an “Immunopure Fab Preparation Kit” (Pierce, 3747 N Meridian Road, PO Box 117, Rockford, Ill. 61105, U.S.A.) according to manufacturers instructions. These Fab fragments were then radiolabelled with Iodine 125 by Amersham Pharmacia Biotech (Pollards Wood, Nightingales Lane, Chalfont St Giles, HP8 4SP United Kingdom).

1 μl of radiolabelled antibody was added to 1 ml of plasma from each of a number of healthy volunteers. After incubation for 4 hours, the components of the plasma were separated by High Performance Liquid Chromatography (HPLC). The HPLC system was an Agilent 1100 system.

The mobile phase used for the HPLC was 0.1M NaCl 0.05M Tris HCl, 0.4% (w/v) Tri-sodium citrate pH 7.5. The stationary phase comprised 2×30 cm BioSep-SEC-S 3000 columns in series (Phenomenex, Queens Avenue, Hurdsfield Industrial Estate, Macclesfield, Cheshire SK10 2BN, United Kingdom). Flow rate was 0.7 ml min⁻¹ and the injection volume was 100 μl.

Fractions of the HPLC eluant were collected using an automated Fraction collector, set to collect one fraction every 20 seconds. Radioactivity was then measured in each fraction using a multiwell scintillation counter.

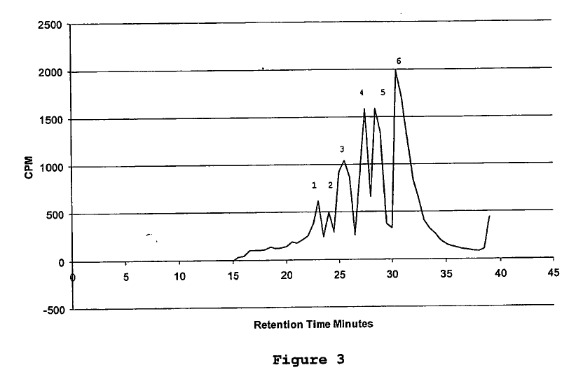

An example of a plot of radioactivity versus time is shown in FIG. 3, where it can be seen that there are several peaks, demonstrating that the radiolabelled antibody fragment has bound to a number of different species within the plasma.

### Example 3

Microtitre Plate Assay for Cellular Factor XIIa

100 μl aliquots of sample were added to wells of a microplate precoated with mAb 2/215. After incubation for 60 minutes, the plates were washed with a borate buffered saline wash solution (pH 7.4). 100 μl of the relevant conjugate (alkaline phosphatase labelled antibody) was added to each well, and the plate was incubated for a further 60 minutes. After washing the plate again, 100 μl of phenolphthalein phosphate substrate was added. After a suitable incubation period, an alkaline Stop solution was added to inhibit further substrate conversion, and the absorbance was recorded at 550 nm.

Method

Blood was collected from a volunteer into two citrate tubes were and the red blood cells were separated by centrifugation at 1000 g for 10 minutes. The plasma from these tubes was pooled to eliminate any collection tube variation. A proportion of the plasma was aliquotted as 1 ml aliquots in Eppendorf tubes) and labeled “cell rich plasma”.

The remainder of the plasma was aliquotted and then centrifuged at high speed (16000 g for 10 minutes) in a microcentrifuge. The supernatant was separated and labeled “cell poor plasma”. The precipitate was then washed by resuspending it in 100 mM Phosphate buffered saline, pH 7.4 (PBS) and centrifuging at 16000 g for 10 minutes, following which the supernatant was discarded. The pelleted material was washed in the same manner a further 2 times, following which it was resuspended in PBS and labeled “washed cells”.

The three samples (cell rich plasma, cell poor plasma and washed cells) were then assayed using the microtitre plate assay described above, in which mAb 2/215 was used to capture cellular Factor XIIa and either labeled mAb 2/215 (2/215 conjugate) or labeled polyclonal antibody (polyclonal conjugate) was used for detection of captured cellular Factor XIIa.

Results

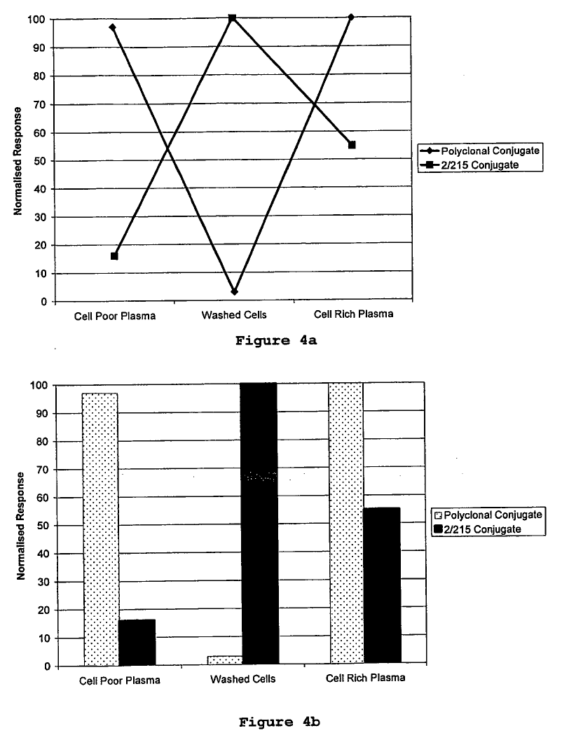

The results obtained are shown in Table 1. The results with the 2/215 conjugate are expressed as absorbances as no standards were available for the dual 2/215 (capture antibody and conjugate antibody) assay. Plots of this data that has been normalized are s shown in FIGS. 4a and 4b.

TABLE 1
Results obtained for different sample types from
microtitre plate XIIa assays with polyclonal and 2/215
conjugates.

| | XIIa MTP | |
|---|---|---|
| | polyclonal | A550 MTP |
| Sample | conj | 2/215 conj |
| Cell poor | 3.5 | 0.209 |
| plasma | | |
| Washed cells | <0.1 | 1.442 |
| Cell Rich | 3.6 | 0.753 |
| plasma | | |

As can be seen from Table 1 and from FIGS. 4a and 4b, with the polyclonal conjugate significant responses are obtained for both the cell rich and cell poor plasmas, but only a minimal response is obtained with the washed cells. In contrast when using the 2/215 conjugate, the maximal response is obtained with the washed cells, whilst the lowest response is obtained for the cell poor plasma.

### Example 4

IMx Assay of Cellular Factor XIIa

The Abbott IMx system is an automated immunoassay analyser designed to run assays using enzyme immunoassay and fluoresence polarisation immunoassay technologies.

The technique used in these Examples is microparticle enzyme immunoassay (MEIA). MEIA technology uses microparticles coated with a capture molecule (in this case an antibody) specific for the molecule being measured. The effective surface area of the microparticles and diffusion distance between analyte and solid phase result in improved assay kinetics, permitting MEIA assays to be completed more rapidly than many other immunoassays. The microparticles along with the bound analyte are separated from the reaction mixture by binding irreversibly to the glass fibre matrix used in the MEIA reaction cell.

The reactants necessary for MEIA assays are
Microparticles coated with a capture molecule (in this case monoclonal antibody 2/215)
Alkaline phosphatase-labeled conjugate (in this case antibodies against activated Factor XII, either polyclonal antibodies or mAb 2/215)
Fluorogenic substrate, 4-methylumbelliferyl phosphate (MUP)
Reaction cell that contains a glass fibre matrix to which immune complex binds.

Other reagents such as a diluent and/or wash solution are also required.

The following is a description of the MEIA Reaction process.
1. The IMx system transfers sample and microparticles (coated with capture molecules) to the incubation well of the reaction cell. During an incubation period, analytes bind to the microparticles, creating an immune complex.
2. The IMx System transfers an aliquot of the immune complex to the inert glass fibre matrix of the reaction cell. The immune complex binds irreversibly to the glass fibre matrix. The IMx washes the matrix to remove unbound materials, and the immune complex is retained by the glass fibres whilst the excess reaction mixture flows rapidly through the large pores in the matrix.
3. The IMx system adds alkaline phosphatase labelled conjugate to the matrix. The conjugate binds to the immune complex to complete the antibody-analyte-conjugate “sandwich”. The IMx washes the matrix again.
4. The IMx system adds the flurogenic substrate 4-methylumbelliferyl phosphate (MUP) to the matrix. The conjugate catalyses the hydrolysis of 4-methyl-umbelliferyl phosphate (MUP) to 4-methylumbelli-ferone (MU)
5. The MEIA optics within the IMx instrument measure the rate at which the fluorescent product (MU) is generated on the glass fibre matrix. The rate at which MU is generated on the matrix is proportional to the concentration of the analyte in the test sample.

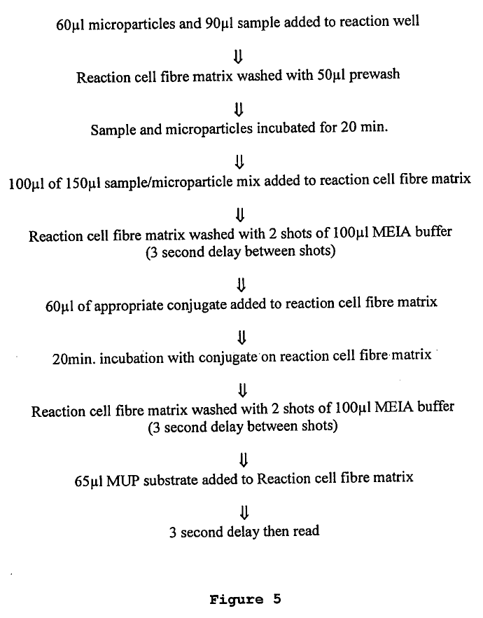

The protocol used for the IMx experiments described below is set out in FIG. 5 of the accompanying drawings.

Method and Results

Blood was collected from a volunteer into 6 citrate tubes and red blood cells were separated by centrifugation at 1000 g for 10 minutes. The plasma from all tubes was pooled to eliminate any collection tube variation. A proportion of the plasma was aliquotted as 1 ml aliquots in Eppendorf tubes and labeled “cell rich plasma”.

The remainder of the plasma was aliquotted and then centrifuged at high speed (16000 g for 10 minutes) in a microcentrifuge. The supernatant was separated and labeled “cell poor plasma”. The precipitate was then washed by resuspending it in 100 mM Phosphate buffered saline, pH 7.4 (PBS) and centrifuging at 16000 G for 10 minutes, following which the supernatant was discarded. The pelleted material was washed in the same manner a further 2 times, following which it was resuspended in PBS and labeled “cell suspension”.

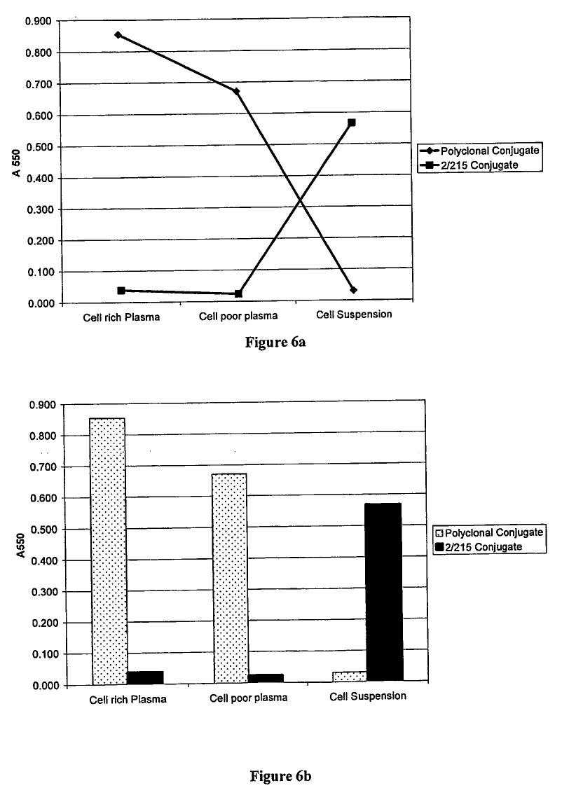

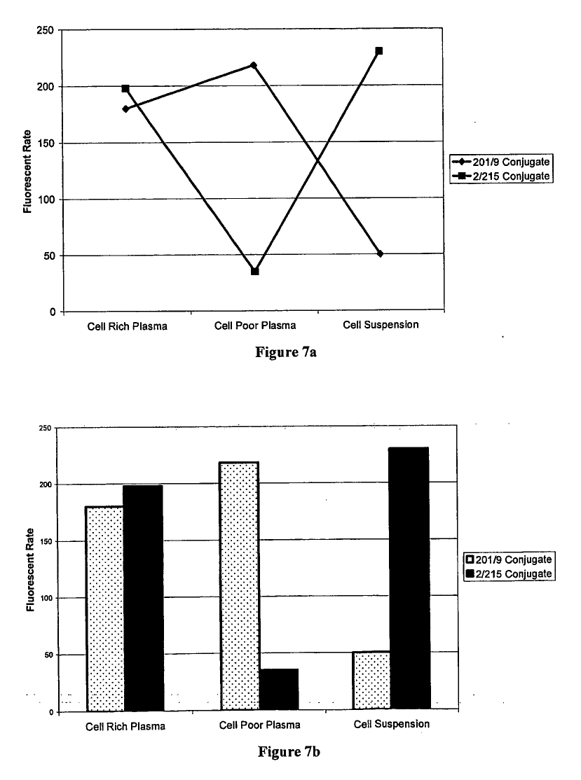

The three samples (cell rich plasma, cell poor plasma and cell suspension) were then assayed using the microtitre plate assay as described in Example 3, using both the 2/215 conjugate and the polyclonal conjugate. The samples were also assayed using an assay for activated Factor XII using the Abbott IMx automated immunoassay instrument. The conjugates used in this case for detection of cellular Factor XIIa were alkaline phosphatase labeled mAbs 201/9 and 2/215, cf Example 3. Plots of data obtained in the microtitre plate assay are shown in FIGS. 6a and 6b, and plots of data obtained in the IMx assay are shown in FIGS. 7a and 7b.

As shown in FIGS. 6a and 6b, in the microtitre plate assay using the polyclonal conjugate, significant responses are obtained for both cell rich and cell poor plasma whilst a minimal response is obtained with the cell suspension, whereas when using the 2/215 conjugate the maximum response was obtained for the cell suspension. Much lower responses were obtained for cell rich and cell poor plasma when using the 2/215 conjugate, with cell poor plasma giving the lowest response.

As shown in FIGS. 7a and 7b, in the IMx assay using the 201/9 conjugate, significant responses are obtained for both cell rich and cell poor plasma whilst a minimal response is obtained with the cell suspension. When the 2/215 conjugate is used in the IMx assay significant responses are seen with the cell suspension and cell rich plasma, with a much reduced response for cell poor plasma.

### Example 5

Flow Cytometric Analysis of Cellular XIIa.

As an alternative means of assessing whether mAb 2/215 was binding to XIIa on cells within plasma, flow cytometry was employed.

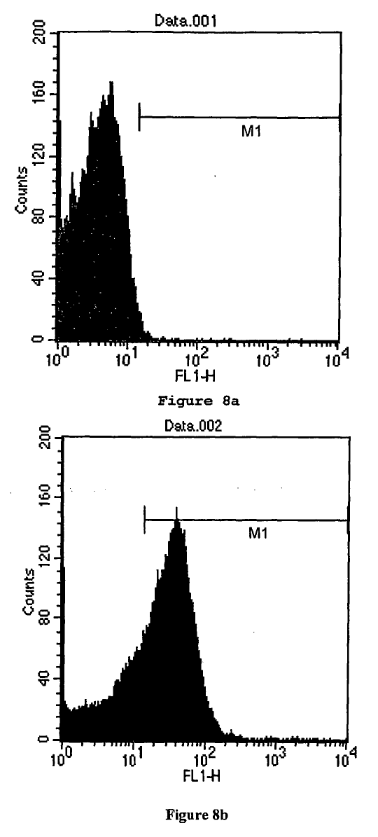

mAb 2/215 was labeled with fluorescein isothiocyanate (FITC). This FITC labeled 2/215 antibody was incubated with cell rich plasma, and along with a control (no labeled antibody added) tested using flow cytometry. The resulting output is shown in FIGS. 8a and 8b. FIG. 8a shows data obtained for the plasma in the absence of labeled antibody, FIG. 8b shows data obtained when plasma was incubated with the labeled antibody.

In FIGS. 8a and 8b, the shift of the peak to the right on the addition of FITC labeled antibody is indicative that the antibody is binding to cells in the plasma sample.

### Example 6

Measurement of Cellular XIIa by Incubation with Radiolabelled Antibody.

A further method of demonstrating and quantitating (determining) cellular XIIa is by the addition of radiolabelled 2/215 antibody to whole blood or plasma samples, separating the cells by centrifugation and measuring the amount of radioactivity bound to this fraction.

Monoclonal antibody 2/215 was labeled with Iodine 125. Blood samples were obtained from 8 volunteers and incubated with radiolabeled antibody. Red blood cells were removed by centrifugation at low centrifugal force (1000 g), and other cells (including platelets and white blood cells) were separated by centrifugation at higher centrifugal force (16000 g). The pelleted cells were washed by resuspending in 100 mM Phosphate buffered saline, pH 7.4 (PBS) and centrifuging at 16000 g for 10 minutes, following which the supernatant was discarded. The pelleted material was washed in the same manner a further 2 times, following which it was resuspended in PBS and labeled “cell suspension”.

Radioactivity associated with this cellular material, and of total amount of antibody bound to XIIa (as opposed to free antibody) was measured, and the proportion of XIIa in the cellular fraction was calculated accordingly.

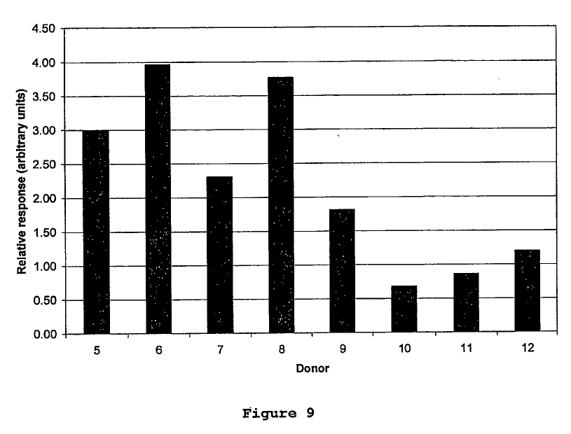

In Table 2, the percentage of added antibody bound to cellular XIIa and the proportion of the antibody bound to XIIa associated with the cellular fraction is shown. It can be seen that there is a significant but variable amount of cellular XIIa. In FIG. 9 is a graphical representation of the relative cellular XIIa concentration for 8 individuals.

TABLE 2
Percentage of added radiolabeled antibody bound
to cellular XIIa and the proportion of the antibody bound
to XIIa associated with the cellular fraction.

| | % of added antibody | Proportion (%) of total |
|---|---|---|
| | bound to cellular | bound antibody in cellular |
| Donor | XIIa | fraction |
| 5 | 2.99 | 23 |
| 6 | 3.96 | 32 |
| 7 | 2.30 | 28 |
| 8 | 3.77 | 34 |
| 9 | 1.81 | 20 |
| 10 | 0.68 | 11 |
| 11 | 0.86 | 12 |
| 12 | 1.20 | 14 |

### Example 7

Cellular XIIa in “Factor XII Deficient” Individuals.

Citrated blood was collected from a “normal” volunteer and from an individual considered totally deficient in Factor XII (demonstrated by a Factor XII antigen ELISA using antibodies available from ERL, 15 Skelty Rd, Swansea, UK) and by measurement of Factor XII using a clotting assay (Griffin, J. H. & Cochrane, C. G., in Methods in Enzymology, Academic Press (New York) 45, 56-65, 1976). Cells were separated by centrifugation at 1000 g for 10 minutes. The plasma from all tubes was pooled for each individual to eliminate any collection tube variation. A proportion of the plasma was aliquotted as 1 ml aliquots in Eppendorf tubes and labeled “cell rich plasma”.

The remainder of the plasma was aliquotted and then centrifuged at high speed (16000 g for 10 minutes) in a microcentrifuge. The supernatant was separated and labeled “cell poor plasma”. The precipitate was then washed by resuspending it in 100 mM Phosphate buffered saline, pH 7.4 (PBS) and centrifuging at 16000 G for 10 minutes, following which the supernatant was discarded. The pelleted material was washed in the same manner a further 2 times, following which it was resuspended in PBS and labeled “cell suspension”.

The samples (cell rich plasma, cell poor plasma and cell suspension) were then assayed using the microtitre plate assay with the 2/215 conjugate and the polyclonal conjugate as described in Example 1. The samples were also assayed using the assay for activated Factor XII using the Abbott IMx automated immunoassay instrument as described in Example 4. The conjugates used in this case for detection of cellular Factor XIIa were peroxidase labeled mAbs 201/9 and 2/215, cf Example 3.

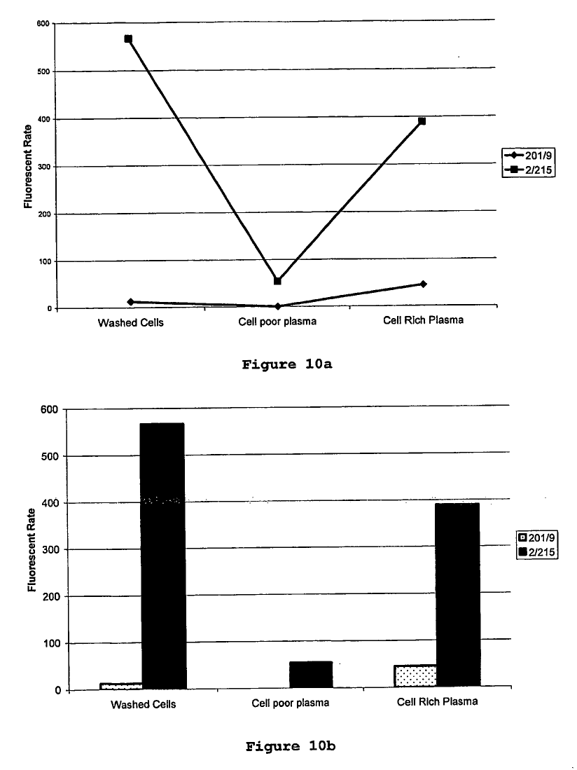

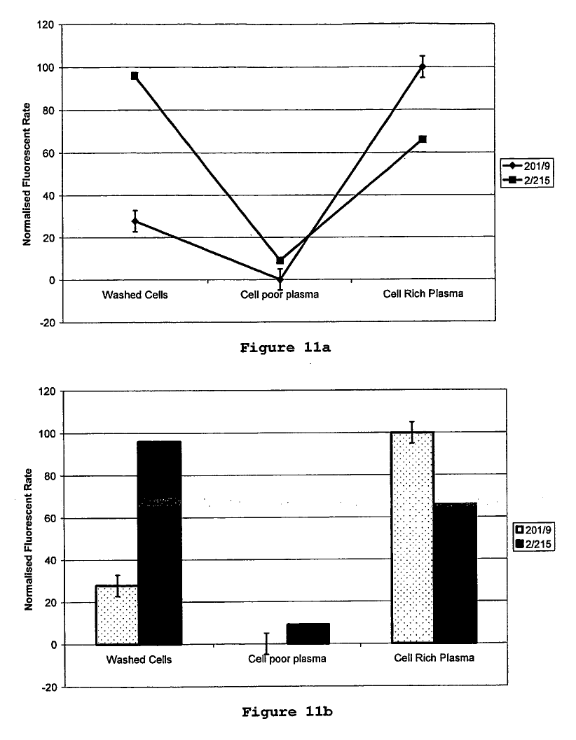

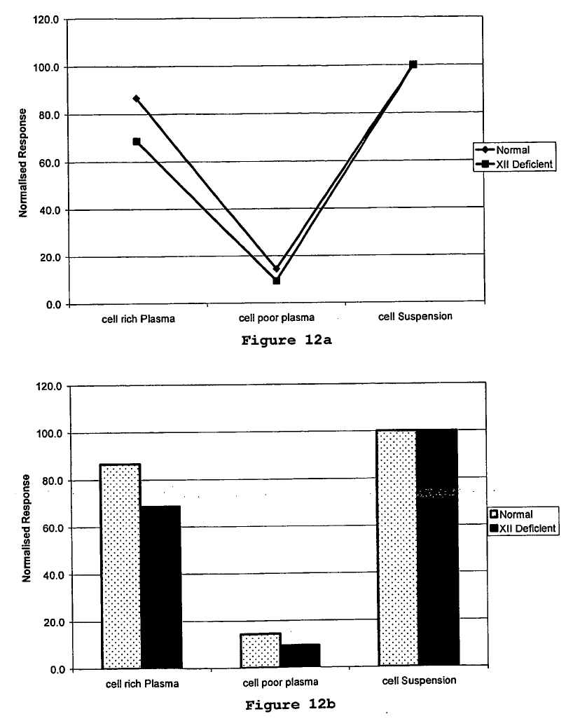

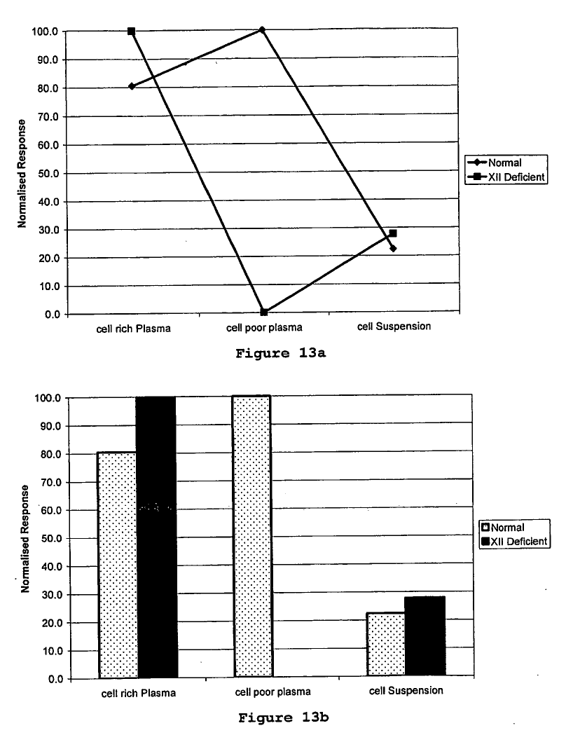

The surprising observation was made that there was a cellular Factor XIIa response from the individual who was “totally Factor XII deficient”, see the data presented in FIGS. 10a, 10b, 11a, 11b, 12a, 12b, 13a and 13b.

Circulating Factor XII originates from the liver. As the “Factor XII deficient “individual has no circulating Factor XII in the aqueous phase, the cell-bound Factor XIIa could not have been formed by adsorption of aqueous phase Factor XII or Factor XIIa, therefore the cellular Factor XII and Factor XIIa must have been produced by another source. It is considered that it is likely is that there is production of Factor XII in other cell lines, for example, lymphocytes or megakaryocytes.

### Example 8

In this example the existence of lipid bound Factor XIIa in plasma was demonstrated by addition to blood plasma of monoclonal antibody 2/215 antibody fragments labelled with a radiotracer (Iodine 125), precipitating the lipoproteins, and assessing the amount of radioactivity associated with the precipitated lipoprotein fraction.

Fab antibody Fragments of antibody 2/215 were prepared using an “Immunopure Fab Preparation Kit” (Pierce, 3747 N Meridian Road, PO Box 117, Rockford, Ill. 61105) according to the manufacturer's instructions. These Fab fragments were then radiolabelled with Iodine 125 by Amersham Pharmacia Biotech (Pollards Wood, Nightingales Lane, Chalfont St Giles, HP8 4SP United Kingdom).

Citrated plasma was obtained from 12 healthy volunteers (6 male and six female).

1 μl of radiolabelled antibody was added to 1 ml of plasma from each of the volunteers. After incubation for 4 hours, the plasma was centrifuged at 12,000 g for 10 minutes to remove cellular components. Lipoproteins were precipitated by the addition to 400 μl of plasma supernatant of 300 μl of a precipitating reagent containing 500 mM NaCl, 215 mM MnCl₂ and 500 U/ml Heparin. After mixing, and incubating for 10 minutes samples were centrifuged at 12,000 g for 10 minutes. The supernatant was removed, and the lipoprotein pellet was washed to remove any residual aqueous phase Factor XIIa, by resuspending the pellet in 1 ml of the precipitation reagent, centrifuging at 12,000 g for 10 minutes and removing the supernatant. After performing this wash procedure three times, radioactivity associated with the pelleted material was measured using a multi-well scintillation counter.

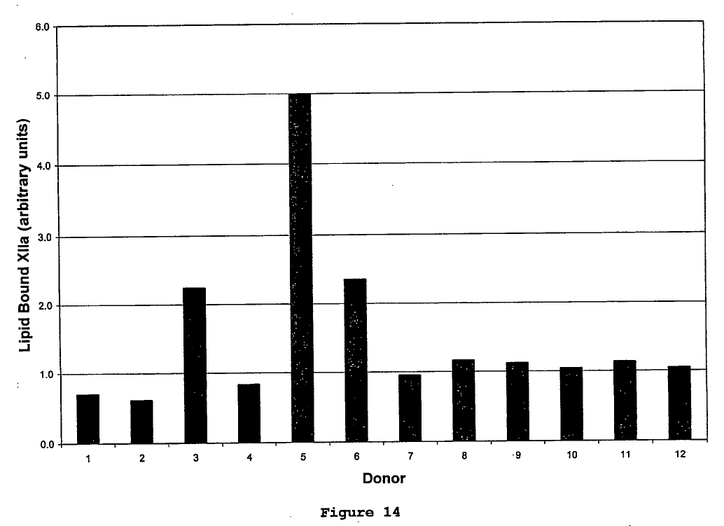

FIG. 14 shows the lipid bound Factor XIIa levels obtained for the 12 volunteers. It can be seen from FIG. 14 that, whilst lipid-bound Factor XIIa is found in all the samples tested, there is considerable variation in concentrations between individuals.

### Example 9

In this example the existence of lipid bound Factor XIIa in plasma was demonstrated by addition to blood plasma of 2/215 antibody fragments labelled with a radiotracer (Iodine 125), precipitating the lipoproteins, and assessing the amount of radioactivity associated with the precipitated lipoprotein fraction.

Fab antibody fragments of antibody 2/215 were prepared using an “Immunopure Fab Preparation Kit” (Pierce, 3747 N Meridian Road, PO Box 117, Rockford, Ill. 61105) according to the manufacturer's instructions. These Fab Fragments were then radiolabelled with Iodine 125 by Amersham Pharmacia Biotech (Pollards Wood, Nightingales Lane, Chalfont St Giles, HP8 4SP United Kingdom).

Citrated plasma was obtained from 64 patients admitted to hospital with chest pain.

5 μl of radiolabelled antibody was added to 1 ml of citrated whole blood from each of the patients. After incubation for 3 hours, the plasma was centrifuged at 16,000 g for 10 minutes to remove cellular components. lipoproteins were precipitated by the addition to 200 μl of plasma supernatant of 500 μl of a precipitating reagent containing 51.54 mM phosphotungstic acid, 0.07 M MgCl₂ adjusted to pH 6.15 with NaOH. After mixing, and incubating for 10 minutes samples were centrifuged at 16,000 g for 10 minutes. The supernatant was removed, and the lipoprotein pellet was washed to remove any residual aqueous phase Factor XIIa, by resuspending the pellet in 1 ml of the precipitation reagent, centrifuging at 16,000 g for 10 minutes and removing the supernatant. After performing this wash procedure three times, radioactivity associated with the pelleted material was measured using a single-well scintillation counter (Lab Logic, St John's House, 131 Psalter Lane, Sheffield, England S11 8UX).

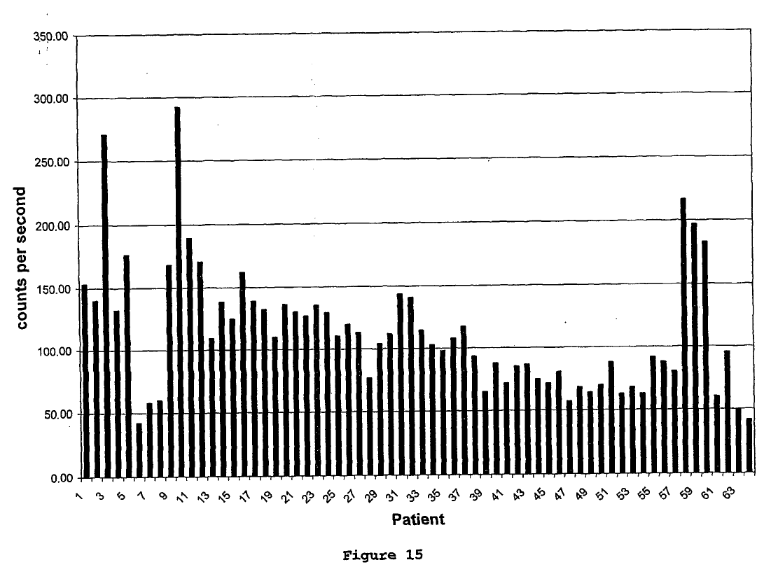

FIG. 15 shows the lipid bound Factor XIIa concentrations obtained for the 64 patients. It can be seen from FIG. 15 that, whilst lipid-bound Factor XIIa is found in all of the samples tested, there is considerable variation in concentrations between individuals.

### Example 10

In this example, a microtitre ELISA immunoassay was used to demonstrate the presence of lipid bound Factor XIIa. Lipoproteins in plasma samples were captured by a antibody directed against a protein present on lipoprotein particles. The presence of Factor XIIa on these lipoproteins was then demonstrated by the addition of alkaline phosphatase labelled mAb 2/215.

Citrated plasma was obtained from 8 healthy volunteers.

100 μl aliquots of citrate plasma were added to wells of a microplate precoated with a goat polyclonal antibody against β-lipoprotein (Sigma, The Old Brickyard, New Road, Gillingham, Dorset, UK). After incubation for 60 minutes, the plates were washed with a borate buffered saline wash solution (pH 7.4). 100 μl of a conjugate containing alkaline phosphatase labelled 2/215 antibody was added to each well, and the plate was incubated for a further 60 minutes. After washing the plate again, 100 μl of phenolphthalein phosphate substrate was added. After a 30 minute incubation period, an alkaline Stop solution was added to inhibit further substrate conversion, and the absorbance was recorded at 550 nm.

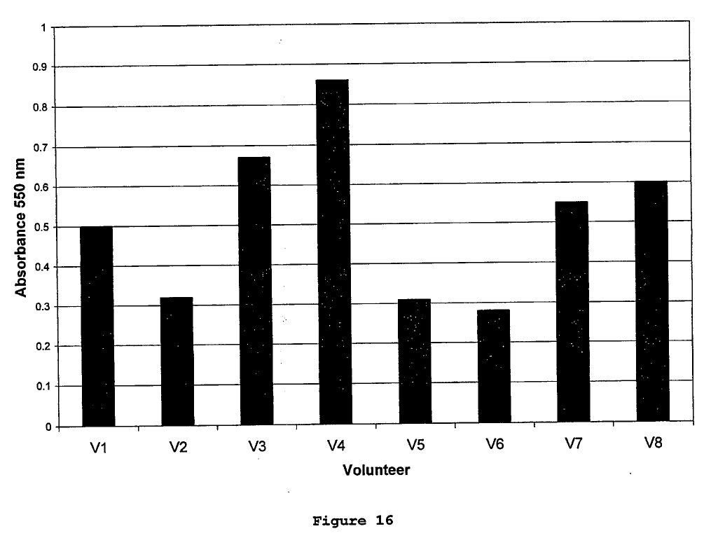

FIG. 16 shows the lipid bound Factor XIIa concentrations, as assessed by the ELISA method described above, obtained for the 8 volunteers. It can be seen from FIG. 16 that, whilst lipid bound Factor XIIa is found in all the samples tested, there is considerable variation in concentrationsd between individuals.

### Example 11

Microtitre Plate Assay of Urinary Factor XIIa

5 normal random urine samples were obtained from healthy male volunteers. These samples were tested for the presence of Factor XIIa using a microtitre plate assay as described below.

100 μl aliquots of sample were added to wells of a microtitre plate precoated with mAb 2/215. After incubation for 60 minutes, the plates were washed with a borate buffered saline wash solution (pH 7.4). 100 μl of conjugate (alkaline phosphatase labelled sheep polyclonal antibody raised against human βXIIa) was added to each well, and the plate was incubated for a further 60 minutes. After washing the plate again, 100 μl of phenolphthalein phosphate substrate was added. After a suitable incubation period, an alkaline Stop solution was added to inhibit further substrate conversion, and the absorbance was recorded at 550 nm. Factor XIIa concentrations in the samples were then calculated by comparison of sample absorbances to those obtained for aqueous samples containing known concentrations of Factor βXIIa. The resultant urinary Factor XIIa concentrations are shown in Table 3.

TABLE 3
Factor XIIa concentrations as assessed by a
microtitre plate assay in random urine samples from
healthy male volunteers.

| | XIIa |
|---|---|
| Volunteer | ng/ml |
| 1 | 0.9 |
| 2 | 1.6 |
| 3 | 1.8 |
| 4 | 1.8 |
| 5 | 1.0 |

### Example 12

IMx Assay of Urinary XIIa

5 Normal random urine samples were obtained from healthy male volunteers. These samples were tested for the presence of Factor XIIa using the IMx assay as described in Example 4 above, using the polyclonal antibody based conjugate. The resulting urinary Factor XIIa concentrations are shown in Table 4.

TABLE 4
Factor XIIa concentrations as assessed by an IMx
assay in random urine samples from healthy male
volunteers.

| | XIIa |
|---|---|
| Volunteer | ng/ml |
| A | 0.5 |
| B | 3.3 |
| B | 2.8 |
| D | 2.3 |
| E | 0.9 |

### Example 13

In this example the existence of urinary Factor XIIa was demonstrated by binding to fluorescently labelled antibody, and separating the antibody that had bound to Factor XIIa from unbound antibody on the basis of molecular weight using high performance liquid chromatography (HPLC).

mAb 2/215 was labelled with Fluorescein Isothiocyanate (FITC) (Pierce, 3747 N Meridian Road, PO Box 117, Rockford, Ill. 61105. U.S.A.) as per the manufacturer's instructions.

The HPLC system consisted of a Waters 1525 Binary HPLC Pump, a Waters 2487 Dual λ Absorbance Detector, and Jasco FP1520 Integral Fluorescence Detector. The mobile phase used for the HPLC was 0.1M NaCl 0.05M Tris HCl, 0.4% (w/v) Tri-sodium citrate pH 7.5. The stationary phase comprised 2×30 cm BioSep-SEC-S 3000 columns in series (Phenomenex, Queens Avenue, Hurdsfield Industrial Estate, Macclesfield, Cheshire SK10 2BN, United Kingdom). Flow rate was 1.0 ml min⁻¹ and the injection volume was 100 μl. Settings for the Jasco Fluorescence detector were. Excitation wavelength 494 nm, emission wavelength 520 nm, Gain 1000, attenuation 1.

Samples run on the HPLC system were the FITC labelled 2/215 alone, a urine sample alone, and urine which had been incubated with FITC labelled 2/215 for 4 hours (250 μl urine plus 1 μl FITC labelled antibody).

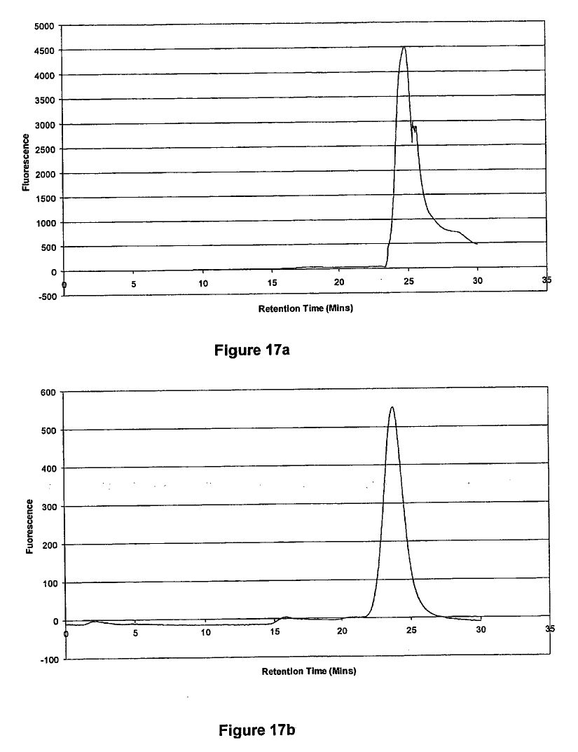

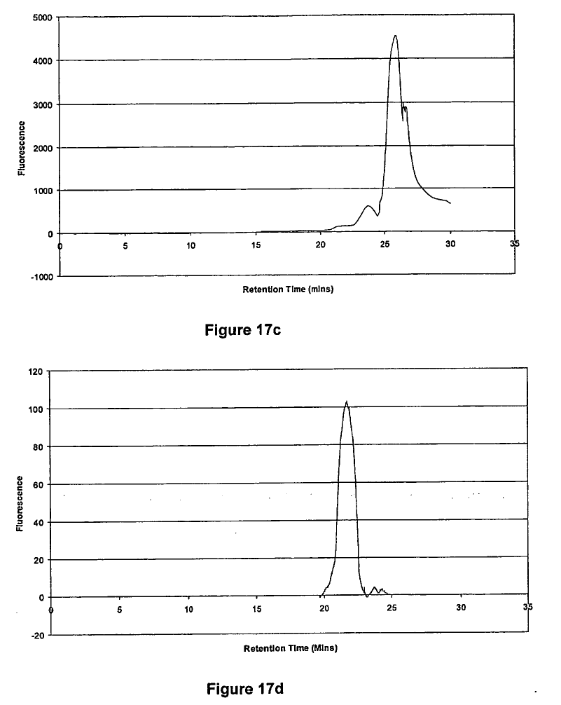

Example plots of fluorescence versus time are shown in FIGS. 17a to 17d.

In FIG. 17a, the trace for the urine sample alone shows that the urine sample exhibits endogenous fluorescence. In FIG. 17b, fluorescence associated with the FITC labelled antibody is observed. In FIG. 17c, urine which has been preincubated with FITC labelled antibody shows a peak additional to those in FIGS. 17a and 17b. This indicates that the FITC labelled antibody is binding to a component in the urine sample. This is further exhibited in FIG. 17d where the signals associated with endogenous fluorescence and the FITC labelled antibody alone have been subtracted, the resultant trace reflecting only the binding of the antibody to urinary Factor XIIa.

### Example 14

In this example the existence of urinary Factor XIIa in plasma was demonstrated by binding to antibody fragments labelled with a radiotracer (Iodine 125), and separating the resultant complexes on the basis of molecular weight using high performance liquid chromatography (HPLC).

The HPLC system consisted of an Agilent 1100 HPLC system.

Fab antibody Fragments of antibody 2/215 were prepared using an “Immunopure Fab Preparation Kit” (Pierce, 3747 N Meridian Road, PO Box 117, Rockford, Ill. 61105, U.S.A.) according to the manufacturer's instructions. These Fab Fragments were then radiolabelled with Iodine 125 by Amersham Pharmacia Biotech (Pollards Wood, Nightingales Lane, Chalfont St Giles, HP8 4SP United Kingdom).

1 μl of radiolabelled antibody was added to 1 ml of urine obtained from healthy volunteers. After incubation for 4 hours, the components of the plasma were separated by High Performance Liquid Chromatography (HPLC).

The mobile phase used for the HPLC was 0.1M NaCl 0.05M Tris HCl, 0.4% (w/v) Tri-sodium citrate pH 7.5. The stationary phase comprised 2×30 cm BioSep-SEC-S 3000 columns in series (Phenomenex, Queens Avenue, Hurdsfield Industrial Estate, Macclesfield, Cheshire SK10 2BN, United Kingdom). Flow rate was 0.7 ml min⁻¹ and the injection volume was 100 μl.

Fractions of the HPLC eluent were collected using an automated fraction collector, set to collect one fraction every 20 seconds. Radioactivity was then measured in each fraction using a multiwell scintillation counter.

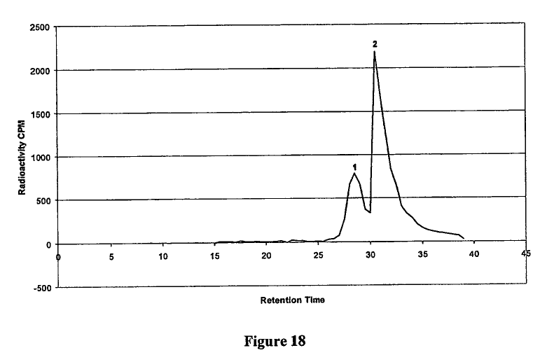

An example of a plot of radioactivity versus time is shown in FIG. 18, where it can be seen that there is a peak additional to that of the unbound antibody fragment demonstrating that the radiolabelled antibody fragment has bound to Factor XIIa present in urine.

### Example 15

In this example the differential response of forms of Factor XIIa in patients undergoing percutaneous transluminal coronary angioplasty (PTCA) is demonstrated.

Samples were taken from patients immediately prior to, immediately after, and 5 days after the coronary artery angioplasty. The samples were then assayed for Factor XIIa using two assays designed to favour measurement of particular forms of XIIa.

In Assay 1, mAb 2/215 was coated on a Nunc (Nunc A/S, Karustrupuej 90, P 0 Box 280, 4000 Roskilde, Denmark) Maxisorb microplate (100 μl of antibody was coated per well) at a concentration of 15 μg ml⁻¹ in a carbonate coating buffer pH. 9.6). 75 μl of plasma with Triton X-100 (Sigma, Fancy Road, Poole, Dorset, England) added to a final Triton concentration of 0.5% (v/v) was added to the wells of the microtitre plate and incubated for 60 minutes at room temperature. After washing the wells of the microtitre plate, 100 μl of conjugate was added. This conjugate comprised monoclonal antibody 201/9 conjugated to alkaline phosphatase. After incubation for 60 minutes the wells of the microtitre plate were again washed and 100 μl of a substrate solution containing phenolphthalein phosphate was added. After incubation for 60 minutes at room temperature the reaction was stopped by the addition of a strongly basic solution (50 g/l sodium carbonate, pH 10.5) and the absorbance at 550 nm was measured.

In Assay 2, antibody 2/215 was coated on a Nunc Maxisorb microplate (100 μl of antibody was coated per well) at a concentration of 2 μg ml⁻¹ in a phosphate coating buffer pH. 7.4). 75 μl of plasma with no Triton X-100 added was added to the wells of the microtitre plate and incubated for 60 minutes at room temperature. After washing the wells of the microtitre plate, 100 μl of conjugate was added. This conjugate comprised polyclonal antibody against anti-Factor XII (Enzyme Research Laboratories, Skelty Road, Swansea, UK) conjugated to alkaline phosphatase. After incubation for 60 minutes the wells of the microtitre plate were again washed and 100 μl of a substrate solution containing phenolphthalein phosphate was added. After incubation for 60 minutes at room temperature the reaction was stopped by the addition of a strongly basic solution (50 g/l sodium carbonate, pH 10.5), and the absorbance at 550 nm was measured.

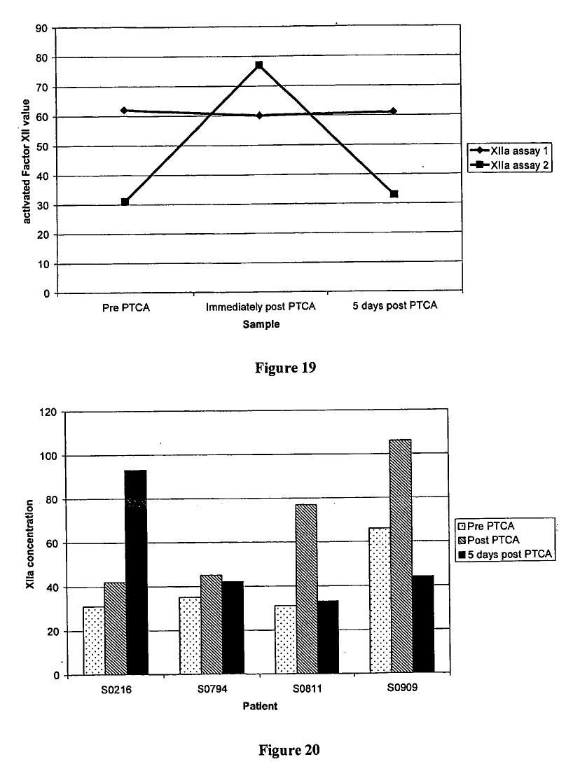

FIG. 19 shows a typical pattern of values obtained from an individual. It can be seen that there is very little change in the concentration of the forms(s) of Factor XIIa preferentially measured by Assay 1 between the pre, immediately post and 5 days post angioplasty samples. This is in marked contrast to the changes in the concentration of the form(s) of Factor XIIa preferentially measured by Assay 2, where a large increase in values is evident immediately post angioplasty, with levels returning to those found pre-angioplasty by day 5.

In FIG. 20 it is shown that the response of particular form(s) of Factor XIIa as measured by Assay 2 varies between individuals undergoing angioplasty. Patient S0216 showed an increase in Factor XIIa immediately post angioplasty and an additional elevation in value was evident with the 5 day sample. Patient S0794 showed only minimal changes in Factor XIIa, whilst patients S0811 and S0909 showed an increase in Factor XIIa immediately post-angioplasty, with 5 day sample giving values similar or even lower than the pre-angioplasty samples. These differences in response reflect the degree of activation of physiological systems involving Factor XIIa and can thus indicate the efficacy of the angioplasty procedure.

### Example 16

In this example the differential response of forms of Factor XIIa in patients undergoing thrombolysis is demonstrated.

Samples were taken from patients immediately prior to, immediately after and 5 days after thrombolytic therapy. The samples were then assayed for Factor XIIa using two assays designed to favour measurement of particular forms of Factor XIIa. The assays used, namely Assay 1 and Assay 2, were as described in Example 15.

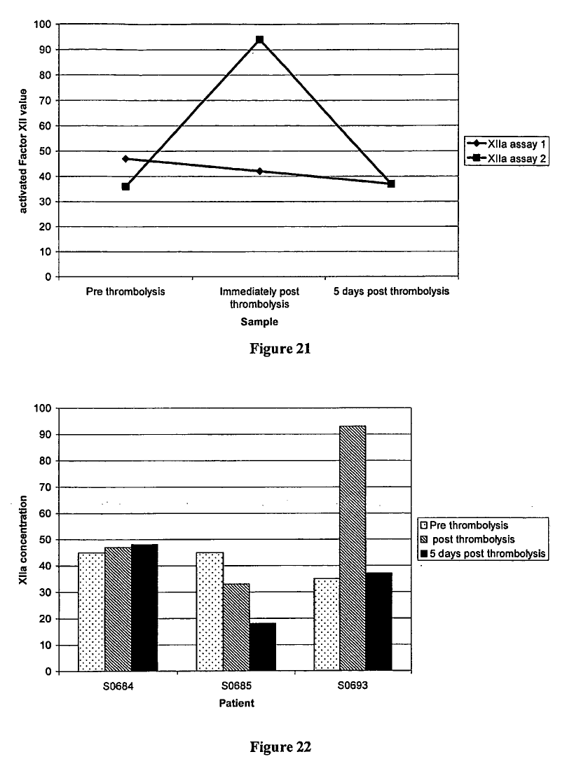

FIG. 21 shows a typical pattern of values obtained from an individual. It can be seen that there is little change in the concentration of the forms of Factor XIIa preferentially measured by Assay 1 between the pre, immediately post and 5 days post thrombolysis samples. This is in marked contrast to the changes in concentration of the forms of Factor XIIa preferentially measured by Assay 2, where a large increase in values is evident immediately post thrombolysis, returning to pre-thrombolysis levels by day 5.

In FIG. 22 it is shown that the response of particular forms of Factor XIIa as measured by Assay 2 varies between individuals undergoing thrombolysis. Patient S0684 showed no change in Factor XIIa upon thrombolysis, Patient S0685 had a progressive decrease in levels immediately post thrombolysis and 5 days after treatment, whereas Patient S0693 had a large increase immediately following thrombolysis, with the level returning to pre-thrombolysis values by 5 days. These differences in response reflect the degree of activation of physiological systems involving Factor XIIa and can thus indicates the efficacy of the thrombolysis procedure. This conclusion is confirmed by the fact that the two patients who showed little difference in values obtained pre and immediately post thrombolysis died within a few days, whereas the patient who exhibited a significant increase in values post-thrombolysis remained event-free for at least 30 days.

### Example 17

This example demonstrates that measurement of particular forms of Factor XIIa provides a prediction of risk of a repeat myocardial infarction or cardiac death in patients admitted to hospital with suspected myocardial infarction and acute coronary syndrome.

Data was obtained on 820 patients admitted to the hospital. Each patient had Factor XIIa measured using several assays, thereby measuring different forms of Factor XIIa. Each assay was studied to ascertain if it provided prediction of the primary clinical endpoints of a subsequent troponin positive event (myocardial infarction) or cardiac death, either during the initial hospitalization period or 30 days after admission.

The prognostic utility of the assays was determined by ranking the Factor XIIa values (from lowest to highest) for each of the assays, each of which preferentially measured different forms of Factor XIIa, and then splitting the population into quartiles i.e. the 205 individuals with the lowest Factor XII concentrations were in the 1^{st} quartile, whilst the 205 individuals with the highest concentrations were in the 4^{th} quartile.

It was found that different forms of Factor XIIa were risk factors for various clinical outcomes, thus different assays, which measure different forms of Factor XIIa provided the best clinical utility for various defined outcomes.

When assessed on the primary clinical end point of a subsequent troponin positive event during the hospitalization period following the initial admission, it was found that the best prognostic indicator was an assay that is considered to measure complexes and/or particles containing multiple Factor XIIa molecules because, inter alia, the sample is not contacted with a detergent, which would disrupt Factor XIIa complexes or Factor XIIa association with particles present in the sample.

The assay used was a microtitre plate format immunoassay in which mAb 2/215 was coated on a Nunc Maxisorb microplate (100 μl of antibody was coated per well) at a concentration of 21 μg ml⁻¹ in a phosphate coating buffer pH. 7.4). 75 μl of plasma with no Triton X-100 added was added to the wells of the microtitre plate and incubated for 60 minutes at room temperature. After washing the wells of the microtitre plate, 100 μl of conjugate was added. This conjugate used the same antibody, mAb 2/215, conjugated to alkaline phosphatase. After incubation for 60 minutes the wells of the microtitre plate were again washed and 100 μl of a substrate solution containing phenolphthalein phosphate was added. After incubation for 60 minutes at room temperature the reaction was stopped by the addition of a strongly basic solution (50 g/l sodium carbonate, pH 10.5), and the absorbance at 550 nm was measured.

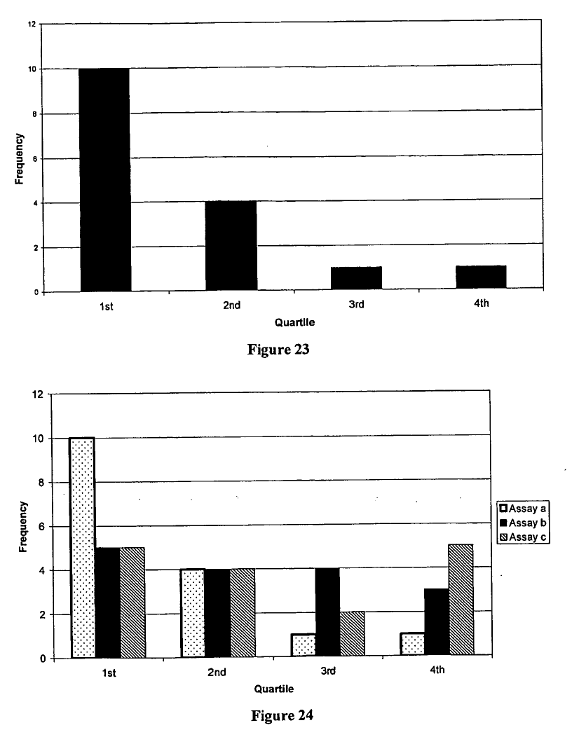

It can be seen from FIG. 23 that individuals with low concentrations of forms of Factor XIIa as measured by the assay, which are considered to be forms of Factor XIIa comprising multiple Factor XIIa molecules, which may be associated as a molecular complex of Factor XIIa molecules and/or may be present on the surface of a particle, for example, a cell or cell remnant or a lipoprotein particle or remnant thereof, are at much increased risk of having a secondary troponin positive event during the initial hospitalization period. It can be seen from FIG. 24 that assays that preferentially measure other forms of Factor XIIa do not provide this clinical utility.

However when the primary clinical end point used was a subsequent troponin positive event following the hospitalization period, but within 30 days of the admission date, it was found that high concentrations of different forms of Factor XIIa ie different from the forms measured by the assay described above, were highly predictive.

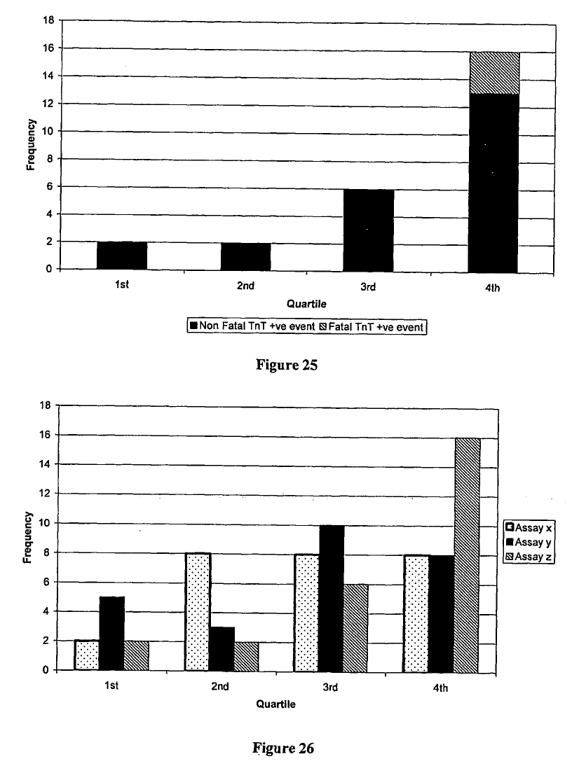

This is shown in FIG. 25 where there are 8 times more events in the 4^{th} quartile than in either of the first two quartiles. Conditions for the microtitre plate assay in FIG. 25 were mAb 2/215 coated on a Nunc Maxisorb microplate (100 μl of antibody was coated per well) at a concentration of 15 μg ml⁻¹ in a carbonate coating buffer pH. 9.6). 75 μl of plasma with Triton X-100 added to a final concentration of 0.5% (v/v) was added to the wells of the microtitre plate and incubated for 60 minutes at room temperature. After washing the wells of the microtitre plate, 100 μl of conjugate was added. This conjugate comprised mAb 201/9 conjugated to alkaline phosphatase. After incubation for 60 minutes the wells of the microtitre plate were again washed and 100 μl of a substrate solution containing phenolphthalein phosphate was added. After incubation for 60 minutes at room temperature the reaction was stopped by the addition of a strongly basic solution (50 g/l sodium carbonate, pH 10.5), and the absorbance at 550 nm was measured. It can be seen from FIG. 26 that assays that preferentially measure other forms of Factor XIIa do not provide this clinical utility.

When death was used as the clinical endpoint, a third assay was found to provide the best clinical utility. This assay appears to measure forms of Factor XIIa related to the risk of the patient dying whether or not the patient has a subsequent troponin positive event.

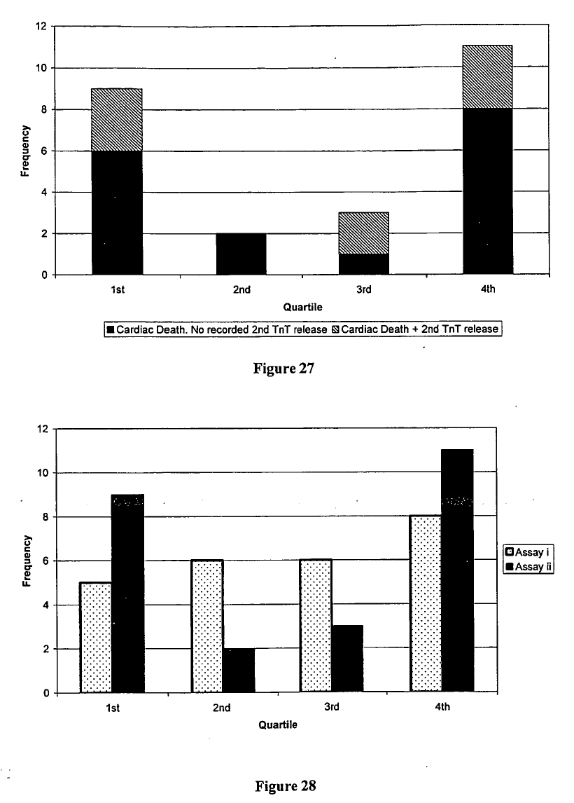

In FIG. 27 it can be seen that there is a U shaped curve, where patients with either lower or higher concentrations of certain forms of Factor XIIa were at significantly increased risk of death compared to those with concentrations of these particular forms closer to the average.

Assay conditions for a microtitre plate assay providing the profile of events in FIG. 27 were antibody 2/215 coated on a Nunc Maxisorb microplate (100 μl of antibody was coated per well) at a concentration of 2 μg ml⁻¹ in a phosphate coating buffer pH. 7.4). 75 μl of plasma with no Triton X-100 added was added to the wells of the microtitre plate and incubated for 60 minutes at room temperature. After washing the wells of the microtitre plate, 100 μl of conjugate was added. This conjugate comprised polyclonal antibody against Factor XII (Enzyme Research Laboratories, Skelty Road, Swansea, UK) conjugated to alkaline phosphatase. After incubation for 60 minutes the wells of the microtitre plate were again washed and 100 μl of a substrate solution containing phenolphthalein phosphate was added. After incubation for 60 minutes at room temperature the reaction was stopped by the addition of a strongly basic solution (50 g/l sodium carbonate, pH 10.5), and the absorbance at 550 nm was measured. It can be seen from FIG. 28 that assays that preferentially measure other forms of Factor XIIa do not provide this clinical utility.

### Example 18

A patient who was admitted to hospital with severe sepsis had Factor XIIa levels measured.

Cellular Factor XIIa levels were measured by incubating 1 ml of citrated whole blood with 5 μl of 2/215 Fab fragment radiolabelled with Iodine 125, and incubating at room temperature for 3 hours, during which time a measurement of the total radioactivity in the sample was measured. Cells were then separated from the plasma by centrifugation at 16,000 g and the plasma was removed. The remaining cells were washed 6 times by the addition of 1 ml of phosphate buffered saline (pH 7.4), mixing, centrifugation at 16,000 g and removal of the supernatant. After these 6 washes, the radioactivity of the remaining cell pellet was assessed and expressed as a percentage of the original total sample radioactivity. This was done to correct for any small variations in amount of radioactive antibody added to the blood sample. As well as using this procedure on the sample from the patient with sepsis, it was also performed on corresponding samples from 100 patients who did not have sepsis.

The cellular Factor XIIa value of the patient with sepsis was 8.20%, whilst the cellular Factor XIIa content of the 100 patients without sepsis ranged from 0.51 to 4.10% (mean 1.50, standard deviation 0.75). Therefore the patient with sepsis had a much higher cellular Factor XIIa value than the control group.

### Example 19

This example demonstrates that measurement of lipid bound Factor XIIa provides a prediction of risk of a repeat myocardial infarction or cardiac death in patients admitted to hospital with suspected myocardial infarction and acute coronary syndrome.

Data was obtained on 160 patients admitted to the hospital with suspected myocardial infarction. Each patient had lipid bound forms of Factor XIIa measured. Results were studied to ascertain if they provided prediction of the primary clinical endpoints of a subsequent troponin positive event or cardiac death, during the 6 months following admission.

The prognostic utility of the assay was determined by ranking the Factor XIIa values (from lowest to highest) and then splitting the population in to quartiles i.e. the 40 individuals with the lowest Factor XII concentrations were in the 1^{st} quartile, whilst the 40 individuals with the highest concentrations were in the 4^{th} quartile. The number of patients in each quartile who had secondary events was then calculated.

Fab antibody fragments of antibody 2/215 were prepared using an “Immunopure Fab Preparation Kit” (Pierce, 3747 N Meridian Road, PO Box 117, Rockford, Ill. 61105) according to the manufacturer's instructions. These Fab Fragments were then radiolabelled with Iodine 125 by Amersham Pharmacia Biotech (Pollards Wood, Nightingales Lane, Chalfont St Giles, HP8 4SP United Kingdom).

Citrated plasma was obtained from 160 patients admitted to hospital with chest pain.

5 μl of radiolabelled antibody was added to 1 ml of citrated whole blood from each of the patients. After incubation for 3 hours, the plasma was centrifuged at 16,000 g for 10 minutes to remove cellular components. lipoproteins were precipitated by the addition to 200 μl of plasma supernatant of 500 μl of a precipitating reagent containing 51.54 mM phosphotungstic acid, 0.07 M MgCl₂ adjusted to pH 6.15 with NaOH. After mixing, and incubating for 10 minutes, samples were centrifuged at 16,000 g for 10 minutes. The supernatant was removed, and the lipoprotein pellet was washed to remove any residual aqueous phase Factor XIIa, by resuspending the pellet in 1 ml of the precipitation reagent, centrifuging at 16,000 g for 10 minutes and removing the supernatant. After performing this wash procedure three times, radioactivity associated with the pelleted material was measured using a single-well scintillation counter (Lab Logic, St John's House, 131 Psalter Lane, Sheffield, England S11 8UX).

Data was then sorted into quartiles as described above, and the number of individuals suffering secondary events was counted within each quartile.

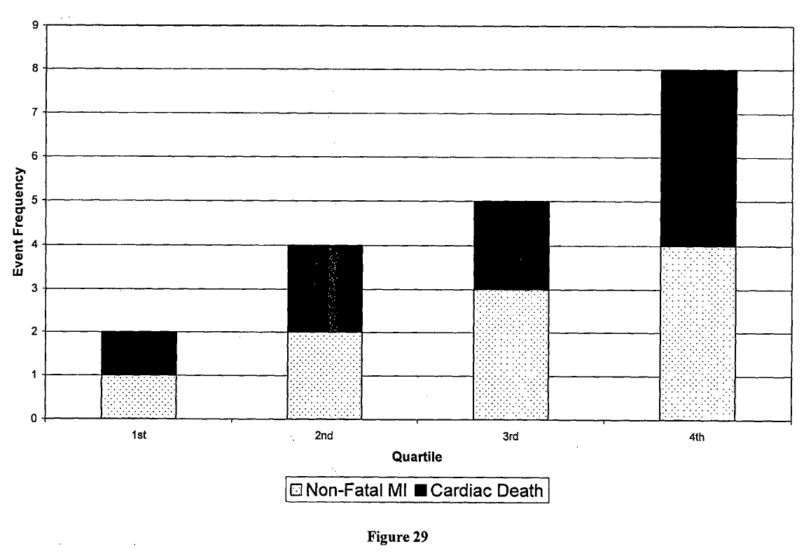

It can be seen from FIG. 29 that increased concentrations of lipid bound Factor XIIa are associated with an increased risk of a secondary event, whether a non-fatal troponin positive event or cardiac death, within 6 months of admission to hospital.

### Example 20

This example demonstrates that concentrations of particular forms of Factor XIIa in urine as measured by incubation with radiolabelled antibody followed by HPLC are increased in patients with renal disease.

24 hour urine samples were obtained from 5 patients with renal failure, and from 5 healthy volunteers. The total collected urine samples from each subject were mixed thoroughly, and 30 ml aliquots were removed and used for analysis.

Fab antibody fragments of antibody 2/215 were prepared using an “Immunopure Fab Preparation Kit” (Pierce, 3747 N Meridian Road, PO Box 117, Rockford, Ill. 61105, U.S.A.) according to manufacturers instructions. These Fab Fragments were then radiolabelled with iodine 125 by Amersham Pharmacia Biotech (Pollards Wood, Nightingales Lane, Chalfont St Giles, HP8 4SP United Kingdom).

1 μl of radiolabelled antibody was added to 1 ml of urine from each volunteer and from each patient. After incubation for 4 hours, the components of the urine were separated by High Performance Liquid Chromatography (HPLC). The HPLC system was an Agilent 1100 system.

The mobile phase used for the HPLC was 0.1M NaCl 0.05M Tris HCl, 0.4% (w/v) Tri-sodium citrate pH 7.5. The stationary phase comprised 1×30 cm BioSep-SEC-S 3000 column and 1×30 cm BioSep-SEC-S 2000 in series (Phenomenex, Queens Avenue, Hurdsfield Industrial Estate, Macclesfield, Cheshire SK10 2BN, United Kingdom). Flow rate was 0.5 ml min⁻¹ and the injection volume was 100 μl.

Radioactivity in the eluate was monitored using an in-line single-well scintillation counter with a high sensitivity iodine 125 detection system (Lab Logic, St John's House, 131 Psalter Lane, Sheffield, England S11 8UX.

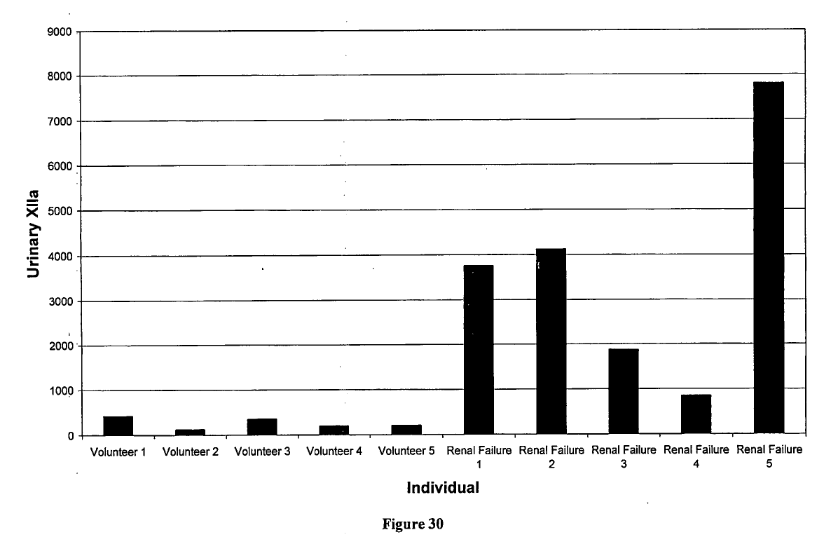

The area of the peak corresponding to Factor βXIIa (based on same retention time as a pure Factor βXIIa incubated with the radiolabelled antibody) was obtained by integration for each of the urine samples. The resulting values are shown in Table 5 and graphically in FIG. 30. It can be seen that values obtained for patients with renal failure are significantly higher than those for the healthy volunteers

TABLE 5
Urinary Factor βXIIa values in 10 urines as
ascertained by incubation with radiolabelled antibody and
HPLC

| | Integrated counts per |
|---|---|
| | second corresponding to |
| Individual | Factor □XIIa peak. |
| Volunteer 1 | 423 |
| Volunteer 2 | 121 |
| Volunteer 3 | 348 |
| Volunteer 4 | 196 |
| Volunteer 5 | 205 |
| Renal Failure 1 | 3756 |
| Renal Failure 2 | 4127 |
| Renal Failure 3 | 1876 |
| Renal Failure 4 | 849 |
| Renal Failure 5 | 7801 |

### Example 21

This example demonstrates that concentrations of particular forms of Factor XIIa in urine as measured by a microtitre plate assay are increased in patients with renal disease.

24 hour urine samples were obtained from 5 patients with renal failure, and from 5 healthy volunteers. Total urine samples from each subject were mixed thoroughly, and 30 ml aliquots were removed and used for analysis.

Antibody 2/215 was coated on a Nunc (Nunc A/S, Karustrupuej 90, P 0 Box 280, 4000 Roskilde, Denmark) Maxisorb microplate (10 μl of antibody was coated per well) at a concentration of 15 μg ml⁻¹ in a carbonate coating buffer pH. 9.6). 75 μl of urine with Triton X-100 added to a final Triton concentration of 0.5% (v/v) was added to the wells of the microtitre plate and incubated for 60 minutes at room temperature. After washing the wells of the microtitre plate, 100 μl of conjugate was added. This conjugate comprised monoclonal antibody 201/9 conjugated to alkaline phosphatase. After incubation for 60 minutes the wells of the microtitre plate were again washed and 100 μl of a substrate solution containing phenolphthalein phosphate was added. After incubation for 60 minutes at room temperature the reaction was stopped by the addition of a strongly basic solution (50 g/l sodium carbonate, pH 10.5) and the absorbance at 550 nm was measured.

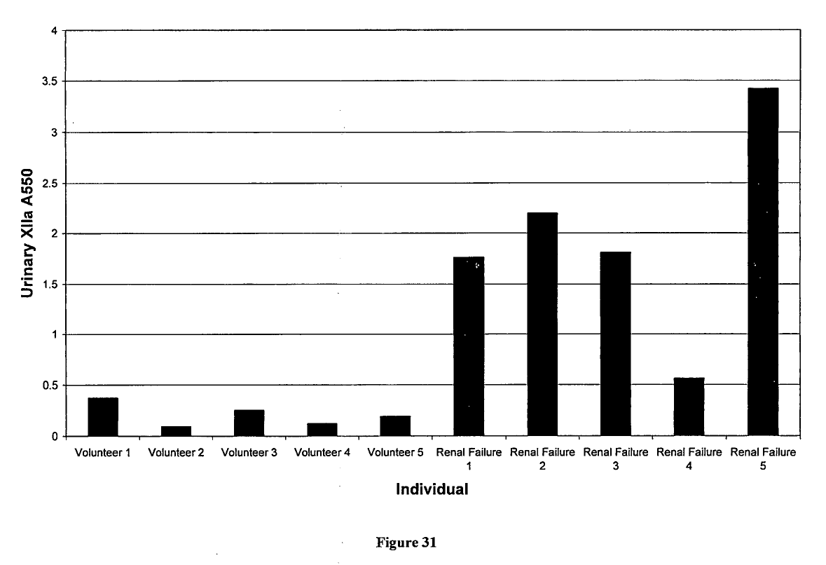

Absorbance values are shown in Table 6, and represented graphically in FIG. 31. It can be seen that individuals with renal disease give significantly higher results than do the healthy volunteers.

TABLE 6
Urinary Factor βXIIa values for 5 healthy
volunteers and 5 individuals with renal disease expressed
as absorbance obtained in a microtitre plate immunoassay.

| Individual | A550 |
|---|---|
| Volunteer 1 | 0.37 |
| Volunteer 2 | 0.09 |
| Volunteer 3 | 0.25 |
| Volunteer 4 | 0.12 |
| Volunteer 5 | 0.19 |
| Renal Failure 1 | 1.76 |
| Renal Failure 2 | 2.20 |
| Renal Failure 3 | 1.81 |
| Renal Failure 4 | 0.56 |
| Renal Failure 5 | 3.42 |

### Example 22

This Example gives a general protocol for producing monoclonal antibodies suitable for use according to the present invention.

The antigen used to raise the antibodies is Factor XII or a fragment thereof. An antigenic fragment of Factor XII may itself be immunogenic or may be too small to be immunogenic, in which case it may be converted into an immunogen, for example, by conjugation to another peptide, for example, as described below. The term “an antigenic fragment of Factor XII” as used herein includes both a fragment, for example, a peptide, and an immunogenic form of such a fragment if it is not itself immunogenic.

An antigenic fragment of Factor XII may be Factor XIIa, for example, Factor α-XII or Factor β-XIIa or a fragment thereof, for example, a peptide that is a fragment of Factor βXIIa that is or that includes at least one antigenic determinant capable of recognising anti-Factor βXIIa.

Methods of preparing immunogens are known to those in the art. Any of these methods may be utilised to render immunogenic or to improve the immunogenicity of Factor XII or antigenic fragment thereof, see also WO90/08835.

For example, Factor βXIIa may be used as the immunogen to raise anti-Factor XIIa monoclonal or polyclonal antibodies. Factor βXIIa may be produced by a method which comprises first isolating Factor XII from fresh or freshly frozen plasma, for example, using a combination of ammonium sulphate precipitation and anion exchange chromatography for example, according to the method described by K. Fujikawa and E. W. Davie (Methods in Enzymol, 1981, 80, 198-211). Methods for converting Factor XII to Factor βXIIa and isolating Factor βXIIa from the resulting mixture are described by K. Fujikawa and B. A. McMullen (Journal of Biol. Chem., 1983, 258, 10924-10933) and B. A. McMullen and K. Fujikawa (Journal of Biol. Chem. 1985, 260, 5328). To obtain Factor βXIIa, Factor XII is generally subjected to limited cleavage, for example, by chemical or enzymatic digestion, for example, using trypsin or a trypsin-like enzyme, generally in a highly diluted form, for example, in a molar ratio of trypsin:Factor XII of 1:500, for example, in a weight ratio trypsin:Factor XII of 1:75 and the cleavage products separated, generally by chromatography.

An antigenic fragment of Factor βXIIa may be produced by degradation of Factor βXIIa by enzymatic or chemical means. For example the disulphide-linked light chain peptide of Factor βXIIa can be obtained by reduction and carboxymethylation of Factor βXIIa and isolation of the fragment by chromatography (K. Fujikawa and B. A. McMullen Journal of Biol. Chem. 1983, 258, 10924). Alternatively, an antigenic fragment of Factor βXIIa may be produced if its amino acid sequence is known, synthetically, as may Factor βXIIa itself. Any of the many known chemical methods of peptide synthesis may be used, especially those utilising automated apparatus.

An antigenic fragment of Factor βXIIa may be produced using the techniques of recombinant DNA technology, as may Factor βXIIa itself. Cool et al, 1985 and 1987, loc. cit. have characterised a human blood coagulation Factor XII cDNA and gene. Recombinant production may be achieved by known methods, see for example, WO90/08835.

Unless specified otherwise, the terms “Factor βXIIa” and “βXIIa” as used herein include antigenic fragments of the Factor βXIIa molecule.

It is preferable, although not essential, that a monoclonal antibody for use according to the present invention shows no significant binding to Factor XII zymogen. In the latter case, the corrected cross-reactivity with Factor XII is, for example, 0.1% or less. A factor to take into consideration in assessing the cross-reactivity of an antibody of the invention with Factor XII is that even “pure” Factor XII preparations are almost inevitably contaminated with small amounts of Factor XIIa (Silverberg and Kaplan, Blood 60, 1982, 64-70). WO90/08835 gives details of methods of assessing the corrected cross-reactivity with Factor XII. Unless specified otherwise, the term “cross reactivity” is used herein to mean the corrected cross reactivity.

Methods used to produce monoclonal antibodies are well known, see for example, Methods in Enzymology, H. Van Vunakis and J. J. Longone (Eds) 1981, 72(B) and ibid, 1983 92 (E).

Monoclonal antibodies may be produced, for example, by a modification of the method of Kohler and Milstein (G. Kohler and C. Milstein, Nature, 1975, 256, 495).

For example, female Balb/C or C57/BI0 mice are immunised by intraperitoneal injection of Factor XII or an antigenic fragment thereof, for example, from 10 to 30 μg, generally 20 μg of Factor βXIIa or a corresponding amount of the other antigen. The Factor βXIIa or other antigen is preferably conjugated to another protein molecule, for example, to a purified protein derivative of tuberculin or, preferably, to bovine thyroglobulin. The conjugation may be carried out, for example, by a carbodiimide method or by using a hetero-bifunctional reagent. The immunogen is generally presented in an adjuvant, preferably complete Freunds adjuvant. This procedure is generally repeated at intervals, generally using the same immunogen in the same dose, for example, at 3 week intervals the mice are boosted with 20 μg of conjugated Factor βXIIa in complete Freunds adjuvant until suitable response levels are observed. A pre-fusion boost is preferably given prior to sacrifice, for example, intravenously 3 days prior to sacrifice.

The antibody response is monitored, for example, by RIA antisera curve analysis using, for example, the desired form of Factor XIIa, for example, any one or more of cellular Factor XIIa, lipid bound Factor XIIa, and Factor XIIa in the form of complexes or associations with other molecules of Factor XIIa or with low or high affinity binding partners, labeled appropriately, for example, ¹²⁵I radiolabelled. In some cases it may be appropriate to use ¹²⁵I radiolabelled Factor XII or a fragment thereof, for example, radiolabelled Factor βXIIa or another Factor βXIIa antigen prepared by the chloramine-T method (P. J. McConahey and F. J. Dixon, Int. Arch. Allergy Appl. Immunol, 1966, 29, 185) at this stage. Purity is confirmed, for example, by using autoradiography, for example, of SDS-PAGE gels run under reducing conditions.

Immune mouse spleen cells are then fused with myeloma cells, for example, NSO mouse myeloma cells, for example in the presence of 40-50% PEG 4,000 or 50% PEG 1500. The cells are then seeded in wells of culture plates and grown on a selective medium. The supernatants are tested for reactivity against the desired form of Factor XIIa, for example, cellular Factor XIIa, lipid bound Factor XIIa, and Factor XIIa in the form of complexes or associations with other molecules of Factor XIIa or with low or high affinity binding partners, for example, by a solid phase enzyme immunoassay, for example, using peroxidase-labelled anti-mouse IgG. All wells showing specificity for the antigen used for testing are generally taken for further secondary screening. The secondary screening consists, for example, of screening all specific antibodies for binding in solution to the appropriate antigen, for example, cellular Factor XIIa, lipid bound Factor XIIa, and Factor XIIa in the form of complexes or associations with other molecules of Factor XIIa or with low or high affinity binding partners. These are preferably titrated to determine the antibody dilution required for 50% B max. Dose-response curves against cold, that is to say non-labelled antigen are generated, and are preferably also generated against Factor XII (if no cross-reactivity with Factor XII is desired). Plasmin and fibronectin may also be used. The extent of cross reaction may be determined according to the following formula:

$$\frac{Weight of Cold Standard Antigen to Achieve 50 \% B max}{Weight of Cross \text{-} Reactant to achieve 50 \% B max} \times 100$$

Those antibodies showing an appropriate level of binding to the desired antigen, for example, having affinity constants of at least 10¹⁰M⁻¹ are generally taken forward for cloning.

Successful clones are generally isotyped. The cells are then preferably sub-cloned by limiting dilution and again screened, for example, using an enzyme immunoassay or radioimmunoassay, for the production of the desired antibodies that bind to the desired forms of Factor XIIa, for example, to any one or more of cellular Factor XIIa, lipid bound Factor XIIa, and Factor XIIa in the form of complexes or associations with other molecules of Factor XIIa or with low or high affinity binding partners. A selected sub-clone from each cloning may also be evaluated with respect to specificity and dose response using a radioimmunoassay or ELISA.

If desired, the antibodies may be screened for those showing a pre-determined apparent cross reactivity to Factor XII, preferably of 1.5% or less, for example 1% or less, for example 0.5% or less, for example, 0.1% or less.

As indicated above, screening against the desired form of Factor XIIa is generally carried out first, but the two or optionally three screens may be carried out in any order. In a screening step, if appropriate, mAb 2/215 or mAb 201/9 may be used as a reference antibody. This is particularly useful if it is desired to obtain a monoclonal antibody that has binding characteristics for particular forms of Factor XIIa the same as or similar to those of mAb 2/215 or 201/9.

Scatchard analysis may be done on the dose-response data to produce values for the affinity constants for each antibody.

Sub-cloned or cloned hybridoma cells may be injected intra-peritoneally into Balb/C mice for the production of ascitic fluid. The immunoglobulin may be precipitated from ascitic fluid, for example, at 4° C. using saturated ammonium sulphate solution (equal volume). The precipitate is preferably purified, for example, it may be centrifuged, dissolved, for example, in 50 mM Tris-HCl buffer pH 7.5 (volume equal to original ascites volume) and then dialysed against the same buffer. The immunoglobulin fraction may then be further purified by anion exchange chromatography, for example, the protein solution may be applied to a Mono-Q anion exchange column (Pharmacia) and eluted using a salt gradient in the same buffer according to the manufacturer's recommendations. The fractions containing immunoglobulin are generally pooled and frozen at −20° C. for storage. Alternatively, hybridoma cells may be grown in culture for antibody production and the antibody isolated essentially as described above for ascites fluid. Alternatively, hybridomas may be cultured in vitro.

Although the hybridomas described herein are derived from mouse spleen cells, the invention is not limited to hybridomas of murine or part-murine origin. Both fusion partners (spleen cells and myelomas) may be obtained from any suitable animal. Recombinant antibodies may be produced. Antibodies may be brought into chimeric or humanized form, if desired. The hybridomas are preferably cultured in vitro.

Deposit of Hybridomas

Monoclonal antibody (mAb) 2/215 is produced by hybridoma 2/215 (BF×11a), deposited at the European Collection of Animal Cell Cultures, Divisional of Biologics, PHLS Centre for Applied Microbiology and Research, Porton Down, Salisbury SP4 0JG, England (known as ECACC) on 16 Jan. 1990 under the deposit number 90011606, and hybridoma 201/9 (ESBT4 1.1), producing monoclonal antibody 201/9, was deposited at ECACC on 18 Jan. 1990 under deposit number 90011893.

1. A method for detecting or determining one or more forms of Factor XIIa in a sample, which comprises carrying out a procedure that is capable of detecting or determining the form or forms of Factor XIIa under investigation in preference to other forms of Factor XIIa.

2. A method as claimed in claim 1, which comprises detecting or determining the form or forms of Factor XIIa under investigation by means of an assay that enables determination of the form or forms of Factor XIIa under investigation in preference to other forms of Factor XIIa.

3. A method as claimed in claim 1, which method comprises separating the form or forms of Factor XIIa under investigation from other forms of Factor XIIa and detecting or determining the separated form or forms of Factor XIIa.

4. A method as claimed in claim 3, wherein the detection or determination of the separated form or forms of Factor XIIa is by means of an assay that enables determination of the form or forms of Factor XIIa under investigation in preference to other forms of Factor XIIa.

5. A method as claimed in claim 1, which comprises contacting the sample with a labeled antibody that is capable of binding to the form or forms of Factor XIIa under investigation and that is optionally also capable of binding to other forms of Factor XIIa, separating the form or forms of Factor XIIa under investigation from other form, and detecting or determining the form or forms of Factor XIIa under investigation.

6. A method as claimed in claim 3, wherein the form or forms of Factor XIIa under investigation is/are separated from other forms of Factor XIIa on the basis of the physical, chemical or immunological properties thereof.

7. A method as claimed in claim 6, wherein the form or forms of Factor XIIa under investigation is/are separated from other forms of Factor XIIa using a chromatographic, flow cytometric or ultracentrifugation procedure, optionally followed by assessment of the enzymatic activity or immunological properties of the separated material.

8. A method as claimed in claim 6, wherein the form or forms of of Factor XIIa under investigation is/are separated by immunoaffinity chromatography using an antibody capable of binding to the form or forms of Factor XIIa under investigation, optionally followed by assessment of enzymatic activity or immunological properties of the separated material.

9. A method as claimed in claim 6, wherein the separation procedure is carried out under conditions such that the form or forms of Factor XIIa is/are not disrupted.

10. A method as claimed in claim 1, wherein the sample is a sample of a body fluid or body tissue.

11. A method as claimed in claim 10, wherein the body fluid is blood, plasma, cerebrospinal fluid, or serum.

12. A method as claimed in claim 10, wherein the body fluid is urine, saliva, or tears.

13. A method as claimed in claim 1, wherein the form of Factor XIIa under investigation is cellular Factor XIIa.

14-16. (canceled)

17. A method as claimed in claim 1, wherein the form of Factor XIIa under investigation is lipid bound Factor XIIa.

18-21. (canceled)

22. A method as claimed in claim 1, wherein the form or forms of Factor XIIa under investigation is any one or more of complexes comprising two or more molecules of Factor XIIa, Factor XIIa associated with low affinity binding partners, and Factor XIIa associated with high affinity binding partners.

23-25. (canceled)

26. A method as claimed in claim 2, wherein the assay is an immunoassay that is capable of detecting or determining the form or forms of Factor XIIa under investigation preferentially relative to other forms of Factor XIIa.

27. A method as claimed in claim 26, wherein the assay comprises the use of an antibody that is capable of binding to the form or forms of Factor XIIa under investigation.

28. A method as claimed in claim 27, wherein the antibody is mAb 2/215 or an analogue thereof, mAb 201/9 or an analogue thereof, or a polyclonal antibody that is capable of binding to Factor XIIa.

29. (canceled)

30. A method as claimed in claim 29, wherein the antibody is radiolabelled.

31-32. (canceled)

33. A method as claimed in claim 26, wherein the sample is a tissue sample and the form or forms of Factor XIIa under investigation is/are detected or determined by immunohistology.

34. A method as claimed in claim 26, wherein the antibody is immobilized on a solid phase as a capture antibody.

35. A method as claimed in claim 34, wherein the antibody immobilized on a solid phase as a capture antibody is mAb 2/215 or an analogue thereof, mAb 201/9 or an analogue thereof, or a polyclonal antibody that is capable of binding to Factor XIIa.

36. A method as claimed in claim 35, wherein the capture antibody is mAb 2/215 (ECACC 90011606) or an analogue thereof.

37. A method as claimed in claim 35, wherein the capture antibody is mAb 201/9 (ECACC 90012512) or an analogue thereof.

38. A method as claimed in claim 34, wherein the solid phase is contacted with the sample and any resulting antigen-antibody complex is detected or determined using a labeled antibody.

39. A method as claimed in claim 38, wherein the labeled antibody is mAb 2/215 or an analogue thereof, mAb 201/9 or an analogue thereof, or a polyclonal antibody that is capable of binding to Factor XIIa.

40-42. (canceled)

43. A method as claimed in claim 1, wherein the procedure enables preferential detection or determination of Factor αXIIa.

44. A method as claimed in claim 1, wherein the procedure enables preferential detection or determination of Factor βXIIa.

45-51. (canceled)

52. A method as claimed in claim 1, wherein the form or forms of Factor XIIa under investigation is any one or more of complexes comprising two or more molecules of Factor XIIa. Factor XIIa associated with low affinity binding partners, and Factor XIIa associated with high affinity binding partners.

53-56. (canceled)

57. A method as claimed in claim 1, wherein the sample has been obtained from a subject having a disease or disorder, undergoing treatment for a disease or disorder, or after having had a disease or disorder or treatment for the disease or disorder.

58. A method as claimed in claim 57, wherein the disease or disorder involves the coagulation system.

59. A method as claimed in claim 57, wherein the disease or disorder involves hemaocoagulation, fibrinolysis, kininogensis, complement activation or angiogenesis, maintaining vascular wholeness and blood pressure, maintaining the constitutive anticoagulant character of the intravascular space, or tissue defence and repair.

60. A method as claimed in claim 57, wherein the disease or disorder is or involves acute or chronic inflammation, shock of any aetiology including septic shock, diabetes, allergy, a thrombo-haemorrhagic disorder, sepsis, spontaneous abortion or an oncological disease.

61. A method as claimed in claim 57, wherein the disease or disorder is or involves intravascular blood coagulation or thromboembolism, a myocardial infarction, acute coronary syndrome or angina.

62. A method as claimed in claim 57, wherein the disease or disorder is or involves thrombosis or stenosis.

63. A method as claimed in claim 57, wherein the disease or disorder is or involves suspected myocardial infarction or acute coronary syndrome.

64. A method as claimed in claim 57, wherein the disease or disorder is or involves sepsis.

65. A method as claimed in claim 57, wherein treatment involves administration of a therapeutic agent and/or involves a surgical procedure.

66. A method as claimed in claim 65, wherein the treatment is coronary artery angioplasty or thrombolysis.

67. A method as claimed in claim 57, wherein a series of samples obtained from a subject are tested.

68. A method as claimed in claim 67, wherein samples are obtained during the course of the disease or disorder.

69. A method as claimed in claim 67, wherein samples are obtained during treatment of the disease or disorder, before treatment is started and/or after treatment has finished.

70. A method for diagnosing, monitoring, or predicting the susceptibility to, progress of, or outcome of a disease or disorder, or of treatment of the disease or disorder in a subject having or suspected of having the disease or disorder, which comprises detecting or determining one or more forms of Factor XIIa in preference to other forms of Factor XIIa in a sample obtained from the subject, and comparing the results obtained for the subject with the results obtained using the same assay for samples obtained from at least any one or more of the following:
(i) subjects having the disease or disorder;
(ii) subjects having the disease or disorder, which subjects were monitored in relation to the progress and/or outcome of the disease or disorder;
(iii) subjects having the disease or disorder and the treatment;
(iv) subjects having the disease or disorder and the treatment, which subjects were monitored in relation to the treatment in relation to the progress and/or outcome of the disease or disorder;
(v) subjects who do not have the disease or disorder;
(vi) the same subject before the onset of the disease or disorder or before the start of the treatment of the disease or disorder; and
(vii) the same subject at an earlier or later stage of the disease or disorder or the treatment of the disease or disorder or before the onset of the disease or disorder.

71-74. (canceled)

75. A method as claimed in claim 70, wherein the sample is from a subject with suspected myocardial infarction, and wherein low levels of particular forms of Factor XIIa are associated with an increased risk of a secondary troponin positive event.

76. (canceled)

77. A method as claimed in claim 70, wherein the sample is obtained from a subject with suspected myocardial infarction, and wherein levels of particular forms of Factor XIIa are associated with an increased risk of death.

78. (canceled)

79. A method as claimed in claim 70, wherein high levels of particular forms of Factor XIIa are associated with sepsis.

80. A method comprising carrying out a series of assays for Factor XIIa on samples obtained from subjects having a disease or disorder or treatment for a disease or disorder, and selecting an assay that provides information on Factor XIIa levels that is relevant to the disease or disorder or the treatment.

81. A method for providing an assay for Factor XIIa suitable for providing information relevant for diagnosing, monitoring, or predicting the susceptibility to, progress of, or outcome of a disease or disorder, or of treatment of the disease or disorder in a subject having or suspected of having the disease or disorder, which comprises carrying out a series of assays for Factor XIIa on samples obtained from subjects having the disease or disorder or the treatment, and determining which assay(s) provide information on Factor XIIa levels that is relevant to diagnosing, monitoring, or predicting the susceptibility to, progress of, or outcome of the disease or disorder, or of treatment of the disease or disorder.

82. A method as claimed in claim 81, comprising comparing the results obtained for Factor XIIa in the samples obtained from subjects having the disease or disorder otr the treatment with the results obtained using the same assay for samples obtained from at least any one or more of the following:
(i) subjects having the disease or disorder;
(ii) subjects having the disease or disorder, which subjects were monitored in relation to the progress and/or outcome of the disease or disorder;
(iii) subjects having the disease or disorder and the treatment;
(iv) subjects having the disease or disorder and the treatment, which subjects were monitored in relation to the treatment in relation to the progress and/or outcome of the disease or disorder;
(v) subjects who do not have the disease or disorder;
(vi) the same subject before the onset of the disease or disorder or before the start of the treatment of the disease or disorder; and
(vii) the same subject at an earlier or later stage of the disease or disorder or the treatment of the disease or disorder or before the onset of the disease or disorder.

83. A method as claimed in claim 70, wherein the assay is an immunoassay comprising an antibody immobilized on a solid phase as a capture antibody, wherein the capture antibody is mAb 2/215 or an analogue thereof, mAb 201/9 or an analogue thereof, or a polyclonal antibody that is capable of binding to Factor XIIa, wherein the solid phase is contacted with the sample and any resulting antigen-antibody complex is detected or determined using a labeled antibody, and wherein the labeled antibody is different from said capture antibody and is mAb 2/215 or an analogue thereof, mAb 201/9 or an analogue thereof, or a polyclonal antibody that is capable of binding to Factor XIIa.

84-87. (canceled)

88. A database comprising the results obtained according to a method as claimed in claim 80.

89. (canceled)

90. A method for diagnosing or monitoring a disease or disorder, or monitoring treatment of the disease or disorder, which comprises detecting or determining Factor XIIa in the urine of a subject having or suspected of having the disease or disorder.

91. A method as claimed in claim 90, wherein the disease is or involves renal function, renal disease or renal damage, or treatment therefore. 92-93. (canceled)
